(19) United States
(12) Patent Application Publication
(10) Pub. No.: US 2003/0153014 A1
(43) Pub. Date: Aug. 14, 2003

(54) Compositions and methods for detecting protein modification and enzymatic activity

(76) Inventors: Li Shen, Potomac, MD (US); Hui Cen, Oakland, CA (US)

Correspondence Address: Peng Chen, Morrison & Foerster LLP, Suite 500, 3811 Valley Centre Drive, San Diego, CA, 92130-2332, US

(21) Appl. No.: 10/356,442

(22) Filed: Jan. 30, 2003

Related U.S. Application Data
(62) Division of application No. 09/678,644, filed on Oct. 3, 2000, now abandoned.
(60) Provisional application No. 60/158,560, filed on Oct. 8, 1999.

(51) Int. Cl. G01N 33/53; G01N 33/542; G01N 33/537; G01N 33/543

(52) U.S. Cl. 435/7.9

(57) ABSTRACT

This invention relates generally to the field of protein modification, e.g., post-translational modification. In particular, the invention provides a method for detecting protein modification profile in a sample, which method comprises: a) contacting a sample containing or suspected of containing a target protein with a capture molecule, or a plurality of capture molecules, immobilized on a solid support, said capture molecule is capable of specifically binding to said target protein, whereby said target protein is immobilized on said solid support; and b) assessing modification status and/or identity of said immobilized target protein. Kits and arrays useful for detecting protein modification are also provided. Arrays, kits and methods useful for detecting enzymatic activities, especially protein modification enzymatic activities, are further provided.

This application claims the benefit of the priority date of the U.S. Provisional Patent Application Serial No. 60/158,560, filed Oct. 8, 1999 under 35 U.S.C. §119(e). The content of the above-referenced application is herein incorporated by reference in its entirety.

### TECHNICAL FIELD

This invention relates generally to the field of protein modification, e.g., post-translational modification. In particular, the invention provides a method for detecting protein modification profile in a sample, which method comprises: a) contacting a sample containing or suspected of containing a target protein with a capture molecule, or a plurality of capture molecules, immobilized on a solid support, said capture molecule is capable of specifically binding to said target protein, whereby said target protein is immobilized on said solid support; and b) assessing modification status and/or identity of said immobilized target protein. Kits and arrays useful for detecting protein modification are also provided. Arrays, kits and methods useful for detecting enzymatic activities, especially protein modification enzymatic activities, are further provided.

### BACKGROUND ART

Westernblot and immunoprecipitation (IP) are the most frequently used procedures in laboratory research lab for analyzing a protein for its expression, molecular weight, degradation, and conformational changes. Westernblot procedures detect the presence of an antigen of interest by an antigen-specific antibody after separating proteins by electrophoresis on an acrylamide gel, transferring separated proteins from the acrylamide gel to a nitrocellulose membrane and immunoblotting. IP is a procedure that is used to study the properties of a specific molecule by immunoprecipitating the molecule from a protein mixture and separating the mixture of immunoprecipitants by electrophoresis. Despite the popular use of Westernblot and IP, these procedures remain time-consuming (requiring a minimum of two days to complete each) and are complicated. Both methods rely on electrophoresis for protein separation, and thus each Westernblot and IP procedure is optimally used for analyzing a single protein or at best a few proteins having different molecular weights. To analyze a few proteins with similar molecular weights, separate Westernblots with antibodies specific to each of the proteins of interest need to be performed, requiring a copy of the identical antigen-bearing nitrocellulose membrane for performing each blot for each antigen. A procedure combining IP with Westernblot may be used to analyze proteins with similar molecular weights for their post-translational modification such as tyrosine phosphorylation using anti-phosphotyrosine antibody. However, each antigen of interest requires its own blot.

A kinase assay can be performed by mixing a substrate of the kinase enzyme with enzyme containing solution for a period of time in the presence of r-labeled ATP followed by electrophoresis to separate the enzyme substrate from the rest of proteins in the mixture. The enzyme activity is reflected by the amount of radioactivity incorporated into the substrate.

ELISA assays are widely used for screening agonists and antagonists for a particular protein, a particular post-translational modification of a protein and a biological activity of a protein. Recently, several ELISA assays have been developed for measuring post-translational modification such as tyrosine phosphorylation. These include an ELISA assay for Heregulin-induced ErbB2 phosphorylation by Sadick et al. Analytical Chemistry, 235:207-214 (1996) and an ELISA assay for VEGF-induced Flk-1/KDR phosphorylation. Many groups also have developed ELISA assay for a specific kinase. For the ELISA assay, the cell lysate from each sample is added into a 96-well plate pre-coated with an antibody against a desired antigen to allow binding of the desired protein onto the surface of 96-well. After the binding, cell lysate is removed from the well and replaced with another antigen-specific antibody that is directly or indirectly conjugated with enzyme. The amount of the antigen of interest is then determined by the activity of the enzyme activity. ELISA assays are designed to screen a large number of samples against a single antigen rather than analyze multiple antigens on a single 96-well plate.

Two-dimensional gel electrophoresis is widely used to analyze genome-wide protein expression and modification. However, the accurate determination of each protein remains difficult since the resolution of the current two-dimensional gel electrophoresis (3000-5000 spots) is far below the total number of cellular proteins. Some protein array technologies have also been developed for gene expression and antibody screening. For these technologies, a library of proteins is immobilized on a two-dimensional array. These proteins are either individually prepared and spotted on a specific area of the array or derived from a nucleic acid array through in situ translation. These antigen arrays are used to screen antibodies, ligands and receptors that interact with the antigen on the arrays.

Recently, efforts have been made to develop antibody microarrays for simultaneous detection of protein expression in clinical analytes such as microbiles and immunoglobulins in the blood. These detection systems focus on the detection of a particular protein expression. A ligand binding mass-sensing assay for quantifying a ligand based on its specific affinity for a chemically modified solid material is described in Silzel et al, Clinical Chem. 44:92036-2043 (1998). The assays provide laser-induced fluorescence detection and can detect microscopic volumes of several different capture reagents. See also, Rowe et al, Anal Chem 71,433-439 (1999) which describes a fluorescence-based immunosensor for simultaneous analysis and detection of clinical analytes: A pattern array of recognition elements is immobilized on the surface of a planar waveguide used to capture analyte present in samples; bound analyte is then quantified by fluorescent-detector molecules.

Therefore, it is an object of the present invention to provide compositions, e.g., arrays and kits, and methods for detecting protein or peptide modification and enzymatic activity.

### DISCLOSURE OF THE INVENTION

The methods, kits, arrays and other compositions of the invention provide for contacting a biologically active or activated sample of proteins with a solid support array of capture molecules specific for target proteins that may or may not be present in the sample. The post-translational modified proteins bound to the capture molecules on the solid support are detected using detection means that specifically recognize the modification moiety. The detection means herein is contacting immobilized target proteins (immobilized by binding with capture molecules that immobilized on the solid support) to a detection molecule that specifically bind to modification moiety, a detection molecule that specifically bind to target protein, or contacting immobilized target proteins to a detection reaction that specifically react with a modification moiety, or physical means that specifically recognize a residue present in the modification moiety. The invention provides a means for detecting protein modifications in order to deduce what biological activity or activities, or biological status are present in a given sample of proteins tested. A major advantage of the invention is that the analysis can be simultaneous for whole groups of proteins in a given sample by analyzing either a single type of protein modification at a time, or a profile of protein modification that indicates some information about the biological activity or status present in the sample. The method is especially useful for generating a profile of protein modification. The principles of the invention are also applied to detecting enzymatic activity in an enzymatically active or activated sample of proteins. The invention can be used for making comparisons between control samples and samples that represent a changed condition. The condition can include for example, disease, disease progression, a disease stage, a developmental stage, drug treatment, chemical treatment, physical change, biological change, different tissues, different animals, different cells, different dosages and any other condition it would be useful to identify and characterize by a comparison of protein modification or enzymatic activity.

Method

In one aspect, the present invention is directed to a method for detecting protein modification of a target protein in a sample, which method comprises: a) contacting a sample containing or suspected of containing a target protein with a capture molecule immobilized on a solid support, said capture molecule is capable of specifically binding to said target protein, whereby said target protein is immobilized on said solid support; and b) assessing modification status and/or identity of said immobilized target protein.

Although the present method can be used to assess the protein modification status of a single target protein at a time, the present method is preferably used in a high-throughput format. For example, the protein modification status (or profile) of a plurality of target proteins can be assessed simultaneously by contacting the plurality of target proteins with an immobilized capture molecule, or a plurality of immobilized capture molecules simultaneously. Alternatively, the protein modification status (or profile) of a single target protein can be assessed simultaneously by contacting a target protein with a plurality of immobilized capture molecules simultaneously. Preferably, the protein modification status (or profile) of a plurality of target proteins can be assessed simultaneously by contacting the plurality of target proteins with a plurality of immobilized capture molecules simultaneously.

The protein modification status (or profile) of any plurality, i.e., group, of target proteins can be assessed by the present method. Preferably, the protein modification status (or profile) of a group of structurally and/or functionally related proteins are assessed.

Any molecule, or complex or combination therefor, that is capable of specifically binding to a target protein, or one or more member(s) of a plurality of target proteins, can be used as the capture molecule in the present method. In one specific embodiment, the capture molecule is capable of specifically binding to both a modified and an unmodified forms of a target protein. In another specific embodiment, the capture molecule is capable of specifically binding to a modified form of the target protein but is not capable of specifically binding to an unmodified form of the target protein. In a preferred embodiment, the capture molecule is an antibody, e.g., a polyclonal antibody, a monoclonal antibody, an antibody fragment retaining its desired binding specificity, or a combination thereof.

Any suitable solid support can be used in the present method. In one example, the solid support can be a silicon, plastic, nylon, glass, ceramic, photoresist, rubber or polymer support. The solid support can be in any kind of suitable geometric forms, e.g., a flat support, a set of sticks, or a set of beads. Exemplary flat supports can comprise a slide, a chip, a filter, or a membrane.

Any protein modification, especially post-translational protein modification, can be assessed by the present method. Exemplary protein modifications that can be assessed by the present method include phosphorylation, acetylation, methylation, ADP-ribosylation, addition of a polypeptide side chain, addition of a hydrophobic group, and addition of a carbohydrate. In one specific embodiment, the phosphorylation to be assessed is phosphorylation on tyrosine, serine, threonine or histidine residue. In another specific embodiment, the addition of a polypeptide side chain to be assessed is the addition of ubiquitin. In still another specific embodiment, the addition of a hydrophobic group to be assessed is the addition of a fatty acid, e.g., myristate or palmitate, addition of an isoprenoid, e.g., farnesyl or genranylgenranyl, or addition of a glycosyl-phosphatidyl inositol anchor, e.g., a carbohydrate group comprises glycosyl.

The present method can be used in different formats. For example, the target protein can be immobilized via the specific interaction between a capture molecule and the peptidic portion of the target protein, and then the protein modification status of the immobilized target protein is assessed. Alternatively, the target protein can be immobilized via the specific interaction between a capture molecule and the modification moiety or the combination of the modification moiety and the peptidic portion of the target protein, and then the identity of the immobilized target protein is assessed. Accordingly, in one specific embodiment, the target protein is first immobilized by a capture molecule that is capable of specifically binding to both a modified and an unmodified forms of a target protein, and then the modification status of the immobilized target protein is assessed by contacting the immobilized target protein with a detection molecule that is capable of specifically binding to the modified target protein but is not capable of specifically binding to the unmodified target protein itself. In this embodiment, the modification status of the immobilized target protein can also be determined by other suitable physical or chemical means. For example, the physical or chemical means can comprise chemical or radioisotopic label of the protein modification moiety. Alternatively, the physical or chemical means can comprise any suitable analytical means, e.g., chromatographic, electrophoretic, protein sequencing, mass spectrometry and NMR means, for detecting the protein modification moiety. In another specific embodiment, the target protein is first immobilized by a capture molecule that is capable of specifically binding to a modified form of the target protein but is not capable of specifically binding to an unmodified form of the target protein, and then the identity of the immobilized target protein is assessed by contacting the immobilized target protein with a detection molecule that is capable of specifically binding to the unmodified target protein itself but is not capable of specifically binding to the modified target protein.

The protein modification status of a target protein, or a plurality of target proteins, in any sample can be assessed by the present method. Preferably, the sample to be assessed is a biological sample.

The protein modification status of any target protein, or any plurality of target proteins, can be assessed by the present method. Preferably, the target protein to be assessed is involved in a biological pathway, belongs to a group of proteins with identical or similar biological function, expressed in a stage of cell cycle, expressed in a cell type, expressed in a tissue type, expressed in an organ type, expressed in a developmental stage, a protein whose expression and/or activity is altered in a disease or disorder type or stage, or a protein whose expression and/or activity is altered by drug or other treatments.

In another aspect, the present invention is directed to a method for identifying biologically distinguishable marker(s) associated with a biosample, which comprises: 1) assessing protein modification profile of a biosample through the above-described method; 2) assessing protein modification profile of a comparable control biosample through the above-described method; and 3) comparing the protein modification profile obtained in step 1) with the protein modification profile obtained in step 2) to identify biologically distinguishable protein modification profile marker(s) associated with said biosample. Preferably, the identified biologically distinguishable protein modification profile marker(s) are indicative of the protein modification profile of the biological source from which the biosample is derived.

Kits and Arrays

In still another aspect, the present invention is directed to a kit for detecting protein modification, which kit comprises: a) a capture molecule immobilized on a solid support, said capture molecule is capable of specifically binding to a target protein; and b) means for assessing modification status and/or identity of said target protein.

In yet another aspect, the present invention is directed to an array of protein capture molecules, which array comprises: a) a solid support; and b) a plurality of capture molecules immobilized on said solid support, wherein each of said molecules is capable of specifically binding to both a modified and an unmodified form of a member protein of a plurality of target proteins. Preferably, the plurality of target proteins comprises a group of structurally and/or functionally related proteins.

In yet another aspect, the present invention is directed to an array of protein capture molecules, which array comprises: a) a solid support; and b) a plurality of capture molecules immobilized on said solid support, wherein each of said molecules is capable of binding to a specific epitope with a modification moiety of a modified protein, e.g., Rb.

Array, Kits and Methods for Detecting Enzymatic Activity

In yet another aspect, the present invention is directed to an array of enzyme substrates, which array comprises: a) a solid support; and b) a plurality of substrates immobilized on said solid support, wherein each of said substrates is a substrate of a member enzyme of a group of structurally and/or functionally related enzymes. Preferably, at least one of the member enzymes catalyzes a protein modification reaction.

In yet another aspect, the present invention is directed to a kit for detecting enzymatic activity, which kit comprises: a) an array comprising a solid support, and a plurality of substrates immobilized on said solid support, wherein each of said substrates is a substrate of a member enzyme of a group of structurally and/or functionally related enzymes; and b) means for assessing activity of each of the member enzymes.

In yet another aspect, the present invention is directed to a method for detecting enzymatic activity in a sample, which method comprises: a) contacting a sample containing or suspected of containing a group of structurally and/or functionally related target enzymes with a plurality of substrates immobilized on a solid support, wherein each of said substrates is a substrate of a member enzyme of said group of target enzymes under conditions suitable for said target enzymes to catalyze enzymatic reactions involving said immobilized substrates; and b) assessing enzymatic activities of said target enzymes. Preferably, at least one of the target enzymes catalyzes a protein modification reaction.

### BRIEF DESCRIPTION OF THE DRAWINGS

FIG. 1 illustrates the principle of the P-Y ProArray. Step 1: Specific capturing antibodies against PTKs are immobilized on a two-dimensional surface. Step 2: PTKs from the cell lysate are captured on the two-dimensional surface at specific positions. Step 3: An enzyme- or a fluorescent-dye-conjugated anti-P-Y antibody binds to P-Y residues in the PTK. Step 4: A chemiluminescent or fluorescent signal is generated to indicate P-Y levels in the PTKs.

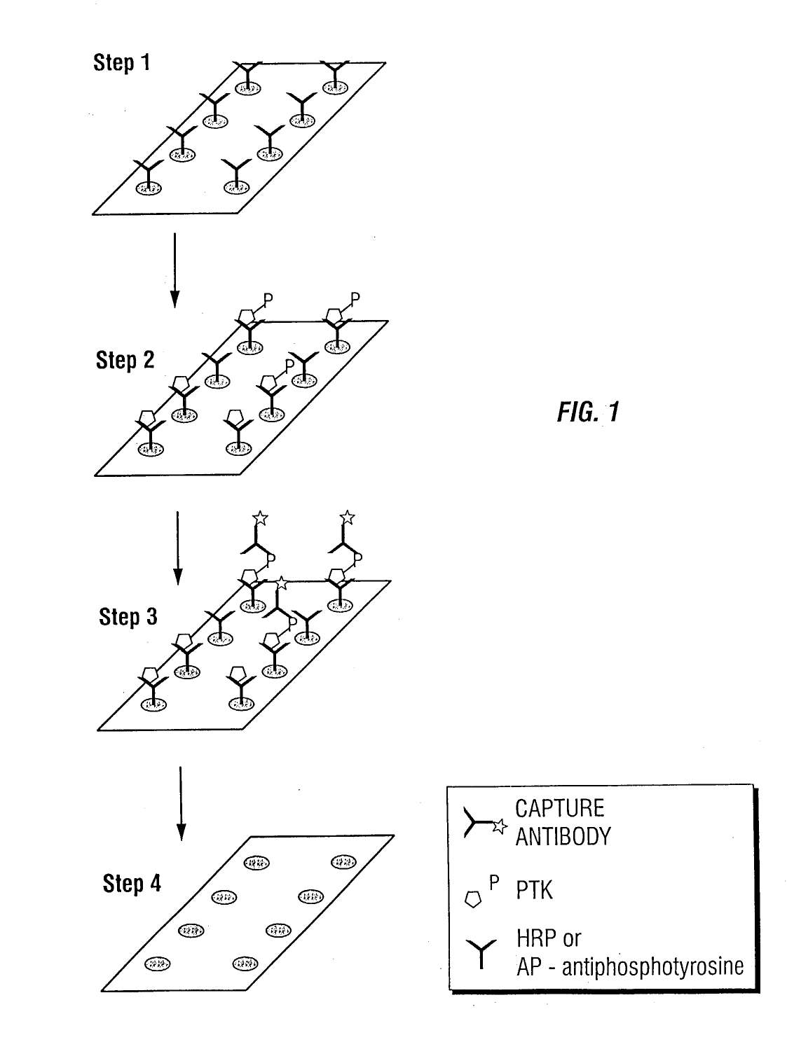

FIG. 2 illustrates the arrangement of an exemplary membrane protein array.

### MODES OF CARRYING OUT THE INVENTION

A. Definitions

Unless defined otherwise, all technical and scientific terms used herein have the same meaning as is commonly understood by one of ordinary skill in the art to which this invention belongs. All patents, applications, published applications and other publications and sequences from GenBank and other databases referred to herein are incorporated by reference in their entirety. If a definition set forth in this section is contrary to or otherwise inconsistent with a definition set forth in applications, published applications and other publications and sequences from GenBank and other data bases that are herein incorporated by reference, the definition set forth in this section prevails over the definition that is incorporated herein by reference.

As used herein, “a” or “an” means “at least one” or “one or more.”

As used herein, “protein” encompasses polypeptide, oligopeptide and peptide.

As used herein, “protein modification” refers to addition of a peptidic or non-peptidic moiety to a protein that cannot be considered as the elongation of the peptidic chain of the protein. The addition of the peptidic or non-peptidic moiety can be in vivo or in vitro. The peptidic or non-peptidic moiety can be added to a pure protein or a protein or peptidic component of a complex containing such protein or peptide. Preferably, “protein modification” refers to post-translational protein modification. Exemplary post-translational protein modification include phosphorylation, acetylation, methylation, ADP-ribosylation, addition of a polypeptide side chain, addition of a hydrophobic group, and addition of a carbohydrate.

As used herein, “capture molecule” refers to a molecule, or complex or combination therefor, that is capable of specifically binding to a target protein, or one or more member(s) of a plurality of target proteins. The capture molecule can be peptides, proteins, e.g., antibodies or receptors, oligonucleotides, nucleic acids, e.g., protein-binding DNA or RNA molecules, vitamins, oligosaccharides, carbohydrates, lipids, small molecules, or a complex thereof.

As used herein, “macromolecule” refers to a molecule that, without attaching to another molecule, is capable of generating an antibody that specifically binds to the macromolecule.

As used herein, “small molecule” refers to a molecule that, without forming homo-aggregates or without attaching to a macromolecule or adjuvant, is incapable of generating an antibody that specifically binds to the small molecule. Preferably, the small molecule has a molecular weight that is about or less than 10,000 daltons. More preferably, the small molecule has a molecular weight that is about or less than 5,000 dalton.

As used herein, “vitamin” refers to a trace organic substance required in certain biological species. Most vitamins function as components of certain coenzymes.

As used herein, “lipid” refers to water-insoluble, oily or greasy organic substances that are extractable from cells and tissues by nonpolar solvents, such as chloroform or ether.

As used herein, a “receptor” refers to a molecule that has an affinity for a given ligand. Receptors may be naturally-occurring or synthetic molecules. Receptors may also be referred to in the art as anti-ligands. As used herein, the receptor and anti-ligand are interchangeable. Receptors can be used in their unaltered state or as aggregates with other species. Receptors may be attached, covalently or noncovalently, or in physical contact with, to a binding member, either directly or indirectly via a specific binding substance or linker. Examples of receptors, include, but are not limited to: antibodies, cell membrane receptors surface receptors and internalizing receptors, monoclonal antibodies and antisera reactive with specific antigenic determinants [such as on viruses, cells, or other materials], drugs, polynucleotides, nucleic acids, peptides, cofactors, lectins, sugars, polysaccharides, cells, cellular membranes, and organelles.

Examples of receptors and applications using such receptors, include but are not restricted to:

a) enzymes: specific transport proteins or enzymes essential to survival of microorganisms, which could serve as targets for antibiotic [ligand] selection;

b) antibodies: identification of a ligand-binding site on the antibody molecule that combines with the epitope of an antigen of interest may be investigated; determination of a sequence that mimics an antigenic epitope may lead to the development of vaccines of which the immunogen is based on one or more of such sequences or lead to the development of related diagnostic agents or compounds useful in therapeutic treatments such as for auto-immune diseases

c) nucleic acids: identification of ligand, such as protein or RNA, binding sites;

d) catalytic polypeptides: polymers, preferably polypeptides, that are capable of promoting a chemical reaction involving the conversion of one or more reactants to one or more products; such polypeptides generally include a binding site specific for at least one reactant or reaction intermediate and an active functionality proximate to the binding site, in which the functionality is capable of chemically modifying the bound reactant [see, e.g., U.S. Pat. No. 5,215,899];

e) hormone receptors: determination of the ligands that bind with high affinity to a receptor is useful in the development of hormone replacement therapies; for example, identification of ligands that bind to such receptors may lead to the development of drugs to control blood pressure; and

f) opiate receptors: determination of ligands that bind to the opiate receptors in the brain is useful in the development of less-addictive replacements for morphine and related drugs.

As used herein, “antibody” includes antibody fragments, such as Fab fragments, which are composed of a light chain and the variable region of a heavy chain.

As used herein, “humanized antibodies” refer to antibodies that are modified to include “human” sequences of amino acids so that administration to a human will not provoke an immune response. Methods for preparation of such antibodies are known. For example, the hybridoma that expresses the monoclonal antibody is altered by recombinant DNA techniques to express an antibody in which the amino acid composition of the non-variable regions is based on human antibodies. Computer programs have been designed to identify such regions.

As used herein the term “assessing” is intended to include quantitative and qualitative determination in the sense of obtaining an absolute value for the amount or concentration of the analyte, e.g., a homocysteine co-substrate, present in the sample, and also of obtaining an index, ratio, percentage, visual or other value indicative of the level of analyte in the sample. Assessment may be direct or indirect and the chemical species actually detected need not of course be the analyte itself but may for example be a derivative thereof or some further substance.

As used herein, “a group of structurally and/or functionally related proteins” refers to a group of proteins, at their natural status, that are structurally linked, located at the same cellular locations, e.g., cellular organelles, located in the same tissues or organs, expressed and/or be functional in the same biological stages, e.g., a particular cell cycle stage or developmental stage, or expressed and/or be functional in the same biological pathway, e.g., a particular metabolism pathway, signal transduction pathway, etc. The “group of structurally and/or functionally related proteins” need only include at least two proteins belonging to the same group. The “group of structurally and/or functionally related proteins” can preferably include more than two proteins belonging to the same group, e.g., a majority of or even all the proteins belonging to the same group.

As used herein, “nutrient or storage protein” refers to a protein that is used by the cell as the nutrient source or storage form for such nutrient. Non-limiting examples of nutrient or storage proteins include gliadin, ovalbumin, casein, and ferritin.

As used herein, “contractile or motile protein” refers to a protein that endows cells and organisms with the ability to contract, to change shape, or to move about. Non-limiting examples of contractile or motile proteins include actin, myosin, tubulin and dynein.

As used herein, “structural protein” refers to a protein that serves as supporting filaments, cables, or sheets to give biological structures strength or protection. Non-limiting examples of structural proteins include keratin, fibroin, collagen, elastin and proteoglycans.

As used herein, “defense protein” refers to a protein that defends organisms against invasion by other species or protect them from injury. Non-limiting examples of defense proteins include antibodies, fibrinogen, thrombin, botulinus toxin, diphtheria toxin, snake venoms and ricin.

As used herein, “regulatory protein” refers to a protein that helps regulate cellular or physiological activity. Non-limiting examples of regulatory proteins include insulin, growth hormones, corticotropin and repressors.

As used herein, “the capture molecule is capable of specifically binding to both a modified and an unmodified forms of a target protein” means that the capture molecule can bind to the target protein through specific interaction between the capture molecule and the peptidic portion of the target protein, and cannot bind to the target protein through specific interaction between the capture molecule and the modification moiety, or the combination of the peptidic portion and the modification moiety of the target protein.

As used herein, “the capture molecule is capable of specifically binding to a modified form of the target protein but is not capable of specifically binding to an unmodified form of the target protein” means that the capture molecule can bind to the target protein through specific interaction between the capture molecule and the modification moiety, or the combination of the peptidic portion and the modification moiety of the target protein, and cannot bind to the target protein through specific interaction between the capture molecule and the peptidic portion of the target protein alone.

As used herein, “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). The sample may also be a mixture of target protein containing molecules prepared in vitro.

As used herein, “a comparable control biosample” refers to a control biosample that is only different in one or more defined aspects from the biosample, and the present methods, kits or arrays are used to identify the effects, if any, of these defined difference(s) between the biosample to be assessed and the control biosample on the protein modification profile of the biosample. For example, the biosample to be assessed and the control biosample can be derived from physiological normal conditions and comparable physiological abnormal conditions, can be subjected to different physical, chemical, physiological or drug treatments, or can be derived from different biological stages, etc.

As used herein, “a group of structurally and/or functionally related enzymes” refers to a group of enzymes, at their natural status, that are structurally linked, located at the same cellular locations, e.g., cellular organelles, located in the same tissues or organs, expressed and/or be functional in the same biological stages, e.g., a particular cell cycle stage or developmental stage, or expressed and/or be functional in the same biological pathway, e.g., a particular metabolism pathway, signal transduction pathway, or act as a regulator for a pathway activation or a biological function, etc. The “group of structurally and/or functionally related enzymes” need only include at least two enzymes belonging to the same group. The “group of structurally and/or functionally related enzymes” can preferably include more than two enzymes belonging to the same group, e.g., a majority of or even all the enzymes belonging to the same group.

As used herein, “expressed in a tissue or organ specific manner” refers to a gene expression pattern in which a gene is expressed, either transiently or constitutively, only in certain tissues or organs, but not in other tissues or organs.

As used herein, “tissue” refers to a collection of similar cells and the intracellular substances surrounding them. There are four basic tissues in the body: 1) epithelium; 2) connective tissues, including blood, bone, and cartilage; 3) muscle tissue; and 4) nerve tissue.

As used herein, “organ” refers to any part of the body exercising a specific function, as of respiration, secretion or digestion.

As used herein, “plant” refers to any of various photosynthetic, eucaryotic multi-cellular organisms of the kingdom Plantae, characteristically producing embryos, containing chloroplasts, having cellulose cell walls and lacking locomotion.

As used herein, “animal” refers to a multi-cellular organism of the kingdom of Animalia, characterized by a capacity for locomotion, nonphotosynthetic metabolism, pronounced response to stimuli, restricted growth and fixed bodily structure. Non-limiting examples of animals include birds such as chickens, vertebrates such fish and mammals such as mice, rats, rabbits, cats, dogs, pigs, cows, ox, sheep, goats, horses, monkeys and other non-human primates.

As used herein, “bacteria” refers to small prokaryotic organisms (linear dimensions of around 1 μm) with non-compartmentalized circular DNA and ribosomes of about 70S. Bacteria protein synthesis differs from that of eukaryotes. Many anti-bacterial antibiotics interfere with bacteria proteins synthesis but do not affect the infected host.

As used herein, “eubacteria” refers to a major subdivision of the bacteria except the archaebacteria. Most Gram-positive bacteria, cyanobacteria, mycoplasmas, enterobacteria, pseudomonas and chloroplasts are eubacteria. The cytoplasmic membrane of eubacteria contains ester-linked lipids; there is peptidoglycan in the cell wall (if present); and no introns have been discovered in eubacteria.

As used herein, “archaebacteria” refers to a major subdivision of the bacteria except the eubacteria. There are three main orders of archaebacteria: extreme halophiles, methanogens and sulphur-dependent extreme thermophiles. Archaebacteria differs from eubacteria in ribosomal structure, the possession (in some case) of introns, and other features including membrane composition.

As used herein, “virus” refers to an obligate intracellular parasite of living but non-cellular nature, consisting of DNA or RNA and a protein coat. Viruses range in diameter from about 20 to about 300 nm. Class I viruses (Baltimore classification) have a double-stranded DNA as their genome; Class II viruses have a single-stranded DNA as their genome; Class III viruses have a double-stranded RNA as their genome; Class IV viruses have a positive single-stranded RNA as their genome, the genome itself acting as mRNA; Class V viruses have a negative single-stranded RNA as their genome used as a template for mRNA synthesis; and Class VI viruses have a positive single-stranded RNA genome but with a DNA intermediate not only in replication but also in mRNA synthesis. The majority of viruses are recognized by the diseases they cause in plants, animals and prokaryotes. Viruses of prokaryotes are known as bacteriophages.

As used herein, “fungus” refers to a division of eucaryotic organisms that grow in irregular masses, without roots, stems, or leaves, and are devoid of chlorophyll or other pigments capable of photosynthesis. Each organism (thallus) is unicellular to filamentous, and possesses branched somatic structures (hyphae) surrounded by cell walls containing glucan or chitin or both, and containing true nuclei.

As used herein, “disease or disorder” refers to a pathological condition in an organism resulting from, e.g., infection or genetic defect, and characterized by identifiable symptoms.

As used herein, “infection” refers to invasion of the body of a multi-cellular organism with organisms that have the potential to cause disease.

As used herein, “infectious organism” refers to an organism that is capable to cause infection of a multi-cellular organism. Most infectious organisms are microorganisms such as viruses, bacteria and fungi.

As used herein, neoplasm (neoplasia) refers to abnormal new growth, and thus means the same as tumor, which may be benign or malignant. Unlike hyperplasia, neoplastic proliferation persists even in the absence of the original stimulus.

As used herein, cancer refers to a general term for diseases caused by any type of malignant tumor.

As used herein, “an immune system disease or disorder” refers to a pathological condition caused by a defect in the immune system. The immune system is a complex and highly developed system, yet its mission is simple: to seek and kill invaders. If a person is born with a severely defective immune system, death from infection by a virus, bacterium, fungus or parasite will occur. In severe combined immunodeficiency, lack of an enzyme means that toxic waste builds up inside immune system cells, killing them and thus devastating the immune system. A lack of immune system cells is also the basis for DiGeorge syndrome: improper development of the thymus gland means that T cell production is diminished. Most other immune disorders result from either an excessive immune response or an ‘autoimmune attack’. For example, asthma, familial Mediterranean fever and Crohn disease (inflammatory bowel disease) all result from an over-reaction of the immune system, while autoimmune polyglandular syndrome and some facets of diabetes are due to the immune system attacking self cells and molecules. A key part of the immune system's role is to differentiate between invaders and the body's own cells—when it fails to make this distinction, a reaction against self cells and molecules causes autoimmune disease.

As used herein, “a metabolism disease or disorder” refers to a pathological condition caused by errors in metabolic processes. Metabolism is the means by which the body derives energy and synthesizes the other molecules it needs from the fats, carbohydrates and proteins we eat as food, by enzymatic reactions helped by minerals and vitamins. There is a significant level of tolerance of errors in the system: often, a mutation in one enzyme does not mean that the individual will suffer from a disease. A number of different enzymes may compete to modify the same molecule, and there may be more than one way to achieve the same end result for a variety of metabolic intermediates. Disease will only occur if a critical enzyme is disabled, or if a control mechanism for a metabolic pathway is affected.

As used herein, “a muscle and bone disease or disorder” refers to a pathological condition caused by defects in genes important for the formation and function of muscles, and connective tissues. Connective tissue is used herein as a broad term that includes bones, cartilage and tendons. For example, defects in fibrillin—a connective tissue protein that is important in making the tissue strong yet flexible—cause Marfan syndrome, while diastrophic dysplasia is caused by a defect in a sulfate transporter found in cartilage. Two diseases that originate through a defect in the muscle cells themselves are Duchenne muscular dystrophy (DMD) and myotonic dystrophy (DM). DM is another ‘dynamic mutation’ disease, similar to Huntington disease, that involves the expansion of a nucleotide repeat, this time in a muscle protein kinase gene. DMD involves a defect in the cytoskeletal protein, dystrophin, which is important for maintaining cell structure.

As used herein, “a nervous system disease or disorder” refers to a pathological condition caused by defects in the nervous system including the central nervous system, i.e., brain, and the peripheral nervous system. The brain and nervous system form an intricate network of electrical signals that are responsible for coordinating muscles, the senses, speech, memories, thought and emotion. Several diseases that directly affect the nervous system have a genetic component: some are due to a mutation in a single gene, others are proving to have a more complex mode of inheritance. As our understanding of the pathogenesis of neurodegenerative disorders deepens, common themes begin to emerge: Alzheimer brain plaques and the inclusion bodies found in Parkinson disease contain at least one common component, while Huntington disease, fragile X syndrome and spinocerebellar atrophy are all ‘dynamic mutation’ diseases in which there is an expansion of a DNA repeat sequence. Apoptosis is emerging as one of the molecular mechanisms invoked in several neurodegenerative diseases, as are other, specific, intracellular signaling events. The biosynthesis of myelin and the regulation of cholesterol traffic also figure in Charcot-Marie-Tooth and Neimann-Pick disease, respectively.

As used herein, “a signal disease or disorder” refers to a pathological condition caused by defects in the signal transudation process. Signal transudation within and between cells mean that they can communicate important information and act upon it. Hormones released from their site of synthesis carry a message to their target site, as in the case of leptin, which is released from adipose tissue (fat cells) and transported via the blood to the brain. Here, the leptin signals that enough has been eaten. Leptin binds to a receptor on the surface of hypothalamus cells, triggering subsequent intracellular signaling networks. Intracellular signaling defects account for several diseases, including cancers, ataxia telangiectasia and Cockayne syndrome. Faulty DNA repair mechanisms are also invoked in pathogenesis, since control of cell division, DNA synthesis and DNA repair all are inextricably linked. The end-result of many cell signals is to alter the expression of genes (transcription) by acting on DNA-binding proteins. Some diseases are the result of a lack of or a mutation in these proteins, which stop them from binding DNA in the normal way. Since signaling networks impinge on so many aspects of normal function, it is not surprising that so many diseases have at least some basis in a signaling defect.

As used herein, “a transporter disease or disorder” refers to a pathological condition caused by defects in a transporter, channel or pump. Transporters, channels or pumps that reside in cell membranes are key to maintaining the right balance of ions in cells, and are vital for transmitting signals from nerves to tissues. The consequences of defects in ion channels and transporters are diverse, depending on where they are located and what their cargo is. For example, in the heart, defects in potassium channels do not allow proper transmission of electrical impulses, resulting in the arrhythmia seen in long QT syndrome. In the lungs, failure of a sodium and chloride transporter found in epithelial cells leads to the congestion of cystic fibrosis, while one of the most common inherited forms of deafness, Pendred syndrome, looks to be associated with a defect in a sulphate transporter.

As used herein, high-throughput screening (HTS) refers to processes that test a large number of samples, such as samples of diverse chemical structures against disease targets to identify “hits” (see, e.g., Broach, et al., High throughput screening for drug discovery, Nature, 384:14-16 (1996); Janzen, et al., High throughput screening as a discovery tool in the pharmaceutical industry, Lab Robotics Automation: 8261-265 (1996); Fernandes, P. B., Letter from the society president, J. Biomol. Screening, 2:1 (1997); Burbaum, et al, New technologies for high-throughput screening, Curr. Opin. Chem. Biol., 1:72-78 (1997)]. HTS operations are highly automated and computerized to handle sample preparation, assay procedures and the subsequent processing of large volumes of data.

For clarity of disclosure, and not by way of limitation, the detailed description of the invention is divided into the subsections that follow.

B. Methods for Detecting Protein Modification

In one aspect, the present invention is directed to a method for detecting protein modification of a target protein in a sample, which method comprises: a) contacting a sample containing or suspected of containing a target protein with a capture molecule immobilized on a solid support, said capture molecule is capable of specifically binding to said target protein, whereby said target protein is immobilized on said solid support; and b) assessing modification status and/or identity of said immobilized target protein.

Although the present method can be used to assess the protein modification status of a single target protein at a time, the present method is preferably used in a high-throughput format. For example, the protein modification status (or profile) of a plurality of target proteins can be assessed simultaneously by contacting the plurality of target proteins with an immobilized capture molecule, or a plurality of immobilized capture molecules, simultaneously. Alternatively, the protein modification status (or profile) of a single target protein can be assessed simultaneously by contacting a target protein with a plurality of immobilized capture molecules simultaneously. Preferably, the protein modification status (or profile) of a plurality of target proteins can be assessed simultaneously by contacting the plurality of target proteins with.

The protein modification status (or profile) of any plurality, i.e., group, of target proteins can be assessed by the present method. Preferably, the protein modification status (or profile) of a group of structurally and/or functionally related proteins are assessed. The “group of structurally and/or functionally related proteins” can be a group of proteins, at their natural status, that are structurally linked, located at the same cellular locations, e.g., cellular organelles, located in the same tissues or organs, expressed and/or be functional in the same biological stages, e.g., a particular cell cycle stage or developmental stage, or expressed and/or be functional in the same biological pathway, e.g., a particular metabolism pathway, signal transduction pathway, or act as a regulator for a pathway activation or a biological function etc.

In a specific embodiment, the group of structurally and/or functionally related proteins is a group of proteins, at their natural status, that are located in the same cellular organelles. Non-limiting examples of such cellular organelles include nucleus, mitochondria, chloroplasts, ribosomes, ERs, Golgi apparatuses, lysosomes, proteasomes, secretory vesicles, vacuoles or microsomes, cytoplasm and other plasms within the such cellular organelles.

In another specific embodiment, the group of structurally and/or functionally related proteins is located in the same tissues or organs. Exemplary tissues include connective, epithelium, muscle or nerve tissues. Exemplary organs include an accessory organ of the eye, annulospiral organ, auditory organ, Chievitz organ, circumventricular organ, Corti organ, critical organ, enamel organ, end organ, external female gential organ, external male genital organ, floating organ, flower-spray organ of Ruffini, genital organ, Golgi tendon organ, gustatory organ, organ of hearing, internal female genital organ, internal male genital organ, intromittent organ, Jacobson organ, neurohemal organ, neurotendinous organ, olfactory organ, otolithic organ, ptotic organ, organ of Rosenmüller, sense organ, organ of smell, spiral organ, subcommissural organ, subfomical organ, supernumerary organ, tactile organ, target organ, organ of taste, organ of touch, urinary organ, vascular organ of lamina terminalis, vestibular organ, vestibulocochlear organ, vestigial organ, organ of vision, visual organ, vomeronasal organ, wandering organ, Weber organ and organ of Zuckerkandl can be manipulated. Exemplary internal animal organs include brain, lung, liver, spleen, bone marrow, thymus, heart, lymph, blood, bone, cartilage, pancreas, kidney, gall bladder, stomach, intestine, testis, ovary, uterus, rectum, nervous system, gland, internal blood vessels, etc can be manipulated.

In still another specific embodiment, the group of structurally and/or functionally related proteins is located in the same body fluid such as blood, urine, saliva, bone marrow, sperm or other ascitic fluids, and subfractions thereof, e.g., serum or plasma.

In yet another specific embodiment, the group of structurally and/or functionally related proteins shares identical or similar structural and/or functional characteristics, such as nutrient or storage proteins, contractile or motile proteins, structural proteins, defense proteins, or regulatory protein.

Any molecule, or complex or combination therefor, that is capable of specifically binding to a target protein, or one or more member(s) of a plurality of target proteins, can be used as the capture molecule in the present method. In one specific embodiment, the capture molecule is capable of specifically binding to both a modified and an unmodified forms of a target protein. In another specific embodiment, the capture molecule is capable of specifically binding to a modified form of the target protein but is not capable of specifically binding to an unmodified form of the target protein. In a preferred embodiment, the capture molecule is an antibody, e.g., a polyclonal antibody, a monoclonal antibody, an antibody fragment retaining its desired binding specificity, or a combination thereof.

The capture molecule can be macromolecules such as peptides, proteins, e.g., antibodies or receptors, oligonucleotides, nucleic acids, e.g., protein-binding DNA or RNA molecules, vitamins, oligosaccharides, carbohydrates, lipids, or small molecules, or a complex thereof.

Any proteins or peptides that are capable of specifically binding to a target protein, or one or more member(s) of a plurality of target proteins, can be used as the capture molecule in the present method. For example, enzymes, transport proteins such as ion channels and pumps, nutrient or storage proteins, contractile or motile proteins such as actins and myosins, structural proteins, defense protein or regulatory proteins such as antibodies, hormones and growth factors can be used.

Any nucleic acids, including single-, double and triple-stranded nucleic acids, that are capable of specifically binding to a target protein, or one or more member(s) of a plurality of target proteins, can be used as the capture molecule in the present method. Examples of such nucleic acids include DNA, such as A-, B- or Z-form DNA, and RNA such as mRNA, tRNA and rRNA.

Any vitamins that are capable of specifically binding to a target protein, or one or more member(s) of a plurality of target proteins, can be used as the capture molecule in the present method. For example, water-soluble vitamins such as thiamine, riboflavin, nicotinic acid, pantothenic acid, pyridoxine, biotin, folate, vitamin B,₂ and ascorbic acid can be manipulated. Similarly, fat-soluble vitamins such as vitamin A, vitamin D, vitamin E, and vitamin K can be used.

Any lipids that are capable of specifically binding to a target protein, or one or more member(s) of a plurality of target proteins, can be used as the capture molecule in the present method. Examples of lipids include triacylglycerols such as tristearin, tripalmitin and triolein, waxes, phosphoglycerides such as phosphatidylethanolamine, phosphatidylcholine, phosphatidylserine, phosphatidylinositol and cardiolipin, sphingolipids such as sphingomyelin, cerebrosides and gangliosides, sterols such as cholesterol and stigmasterol and sterol fatty acid esters. The fatty acids can be saturated fatty acids such as lauric acid, myristic acid, palmitic acid, stearic acid, arachidic acid and lignoceric acid, or can be unsaturated fatty acids such as palmitoleic acid, oleic acid, linoleic acid, linolenic acid and arachidonic acid.

Any suitable solid support can be used in the present method. In one example, the solid support can be a silicon, plastic, nylon, glass, ceramic, photoresist, rubber or polymer support. The solid support can be in any kind of suitable geometric forms, e.g., a flat support, a set of sticks, or a set of beads. Exemplary flat supports can comprise a slide, a chip, a filter, or a membrane. Solid supports and methods for immobilizing desired capture molecules on the solid supports that are known in the art can be used. Preferably, solid supports and methods for immobilizing desired capture molecules on the solid supports that are described in the following Section C can be used in the present method.

Any protein modification, especially post-translational protein modification, can be assessed by the present method. Exemplary protein modifications that can be assessed by the present method include phosphorylation, acetylation, methylation, ADP-ribosylation, addition of a polypeptide side chain, addition of a hydrophobic group, and addition of a carbohydrate. In one specific embodiment, the phosphorylation to be assessed is phosphorylation on tyrosine, serine, threonine or histidine residue. In another specific embodiment, the addition of a polypeptide side chain to be assessed is the addition of ubiquitin. In still another specific embodiment, the addition of a hydrophobic group to be assessed is the addition of a fatty acid, e.g., myristate or palmitate, addition of an isoprenoid, e.g., farnesyl or genranylgenranyl, or addition of a glycosyl-phosphatidyl inositol anchor, e.g., a carbohydrate group comprises glycosyl.

Phosphorylation

Phosphorylation can include phosphorylation of a tyrosine, serine, threonine or histidine. Antibodies that can be used to detect these modifications can include phosphotyrosine-specific antibody, phosphoserine-specific antibody, phosphoserine-specific antibody, and phospho-threonine-proline antibody, for example. Antibodies that can be used to detect these modifications also include an antibody specific to a phosphorylated residue of a protein such as phosphorylated c-Jun at Ser 73. Among various post-translational modifications, protein phosphorylation was found to be the most common mechanism for switching a protein from its active state to an inactive state. The protein phosphorylation includes tyrosine phosphorylation, serine/threonine phosphorylation and histidine phosphorylation. The phosphorylation of p44/42 MAP Kinase (Thr202/Tyr204) and MEKI/2 (Ser217/221) has been found to contribute to the activation of mitogenic signal pathway. The phosphorylation of SAPK/JNK (Thr183/Tyr185), p38 MAP kinase Thr180/Tyr182, MKK3/MKK6 (Ser189/207), SEK1/MKK4 (Thr223) has been found to contribute to the activation of stress signal pathway. The phosphorylation of Akt (Ser473), Bad (Ser112/136) and p70 S6 Kinase (Ser411, Thr421/Ser424) has been found to promote cell survival and prevent cell apoptosis.

The phosphorylation of pathway-specific transcription factors also can serve as a reliable marker for pathway activation. For example, phosphorylation of ikb-a indicates activation of NFkB signal pathway ( ); phosphorylation of ELK1, CREB, Ets1, Ets2, CBP, PEA₃, p₉₀rSk and CEBP indicates activation of mitogenic/differentiation signal pathway ( ); phosphorylation of c-Jun, Elk1, ATF2, c-myc, SAP1a and PEA3 indicates activation of cytoskeletal organization signal pathway ( ); phosphorylation of ATF1, ATF2, Elk1, Max, CHOP, CREB, SAP1a and MAPKAPK-2 indicates activation of apoptosis/stress signal pathway. Several ErbB family receptors are implicated in tumor formation and these receptors are activated through self tyrosine phosphorylation.

A protein array for tyrosine phosphorylation can contain antibodies that can specifically capture proteins whose tyrosine phosphorylated form are of interest. After capturing those proteins on the membrane, an antibody against phosphorylated tyrosine is used to detect the amount of phosphorylated form of each protein. A protein array for tyrosine phosphorylation can be designed for analyzing a groups of proteins involved in mitogenic signal pathway, and can contain antibodies against EGF receptor, PDGF receptor, SOS, Src, p44/4² MAP Kinase. Another protein array for tyrosine phosphorylation can contain antibodies against all four members of ErbB family receptors: EGFR, ErbB-2, ErbB-3 and ErbB-4.

To monitor activation state of mitogenic signal pathway, stress signal pathway and a pptosis/survival signal pathway, a protein array for phosphorylation can be designed for analyzing phosphorylation state of pathway-specific kinases. These kinases can include p44/42 MAP Kinase, MEK1/2, SAPK/JNK, p38 MAP kinase, MKK3/MKK6, SEK1/MKK4, Akt, Bad and p70 S6 Kinase. Alternatively, a protein array for phosphorylation can be designed for analyzing phosphorylation state of pathway-specific transcription factors as listed above.

Acetylation

Acetylation can be detected by use of an acetylated-lysine antibody. Acetylation of p53 is associated with a change of its transcriptional activity after DNA damage. Acetylation of histone H3 is increased in response to DNA damage and mitogen stimulation. A protein array comprising acetylation can be used to simultaneously analyze multiple proteins for their acetylation status in a single assay. Such an array can include capture molecules for p53 and various histones including histone 1, 2A, 2B, 3 and 4, and using acetylation detection antibodies the modification on these capture molecules can be detected.

Methylation

Methylation specific antibodies can be used to detect proteins having a methylation on one or more amino acids of a polypeptide sequence of the protein. Detecting methylation may be useful in a variety of biological contexts, for example, in human neutrophils and other cell types, Ras-related guanosine triphosphate-bin ding proteins are directed toward their regulatory targets in membranes by a series of posttranslational modifications that include methyl esterification of a carboxyl-terminal prenylcysteine residue. The amount of carbosyl methylation of Ras-related proteins increased in response to the chemoattractant N-formyl-methionyl-leucyl-phenylalanine (FMLP). Activation of Ras-related proteins by guanosine-5′-O-(3-thiotriphosphate) has a similar effect and induced translocation of p22rac2 from cytosol to plasma membrane. Inhibitors of prenylsysteine carbosyl methylation effectively block neutrophil responses to FMLP. A protein array for Ras-related proteins for their methylation can be used to simultaneously analyze methylation status of multiple Ras-related proteins in a single assay. These Ras-related proteins can include, for example, H-Ras, K-Ras, R-Ras, RhoA, Rac1, Rac2, Ra1, etc. Antibodies (capture molecules) against each of Ras-related proteins are spotted each on its own specific area of a protein array. The Ras-related proteins of a protein sample are captured and then a detection antibody specific for methylation (an anti-methylation antibody) contacts the captured molecules to detect those proteins that have been methylated.

ADP-Ribosylation

ADP-ribosylation specific antibodies can be used to detect proteins having an ADP ribosylation modification. Detecting ADP ribosylation can be useful in a variety of biological contexts, including, for example in the case of when cholera toxin induces activation of adenylate cyclase from small intestinal epithelium. The activation is associated with ADP-ribosylation of a number of proteins including Gs alpha subunit and a 40kd, a 45 kd and a 47 kd proteins located in the bruch-border membrane. A specialized ADP-ribosylation protein array can be made for studying ADP ribosylation of these cholera toxin related proteins. Antibodies (capture molecules) against each of these proteins can be spotted each on its own specific area of a protein array. The protein array is used to capture these proteins from a sample of proteins and the captured proteins are detected for their ADP ribosylation through contact with an anti-ADP-ribosylation antibody (the detection molecule). Another example of use of a protein array for ADP ribosylation is in the case of poly-ADP ribosylation of histones which is found to increase significantly in mitogen-activated lymphoid cells. A specialized histone poly-ribosylation protein array can be used to simultaneously analyze poly-ribosylation of various histones. Such a protein array can contain antibodies (capture molecules) against various histones such as, for example, histone 1, histone 2A, histone 2B, histone 3 and histone 4. The protein array is used to capture various histones from a sample of proteins and the target proteins are detected for their ADP ribosylation through contact with an anti-ADP-ribosylation antibody or anti-poly-ADP ribosylation antibody.

Polypeptide Chain Addition

An example of addition of a polypeptide chain is ubiquitination. Detection of ubiquitination on a target protein can be made using an ubiquitin-specific antibody or polyubiquitin-specific antibody for example. Ubiquitination involves the covalent attachment of ubiquitin, an evolutionary highly conserved 76-amino acid polypeptide which is abundantly present in all eukaryotic cells to the 1-amino group of one or more lysine side chains of target proteins. Ubiquitination-dependent degradation is involved in the degradation of proteins that are either damaged or no longer needed. Ubiquination plays important role in cell regulation and signal transduction. Protein ubiquitination can be a reversible process and is controlled by two classes of enzymes: ubiquitin conjugating enzymes and deubiquitinating enzymes. Recently, ubiquitination is found to be responsible for the cell cycle-specific degradation of cyclins and cytokine-induced breakdown of the transcription factor inhibitor IkB. In addition, ubiquitination is responsible for the degradation of several transcriptional factors including c-Jun, ATF2, Jun B and p53. Phosphorylation of these transcriptional factors prolongs their half-life significantly by blocking their ubiquitination. A protein array for ubiquitination can be designed for analyzing a group of transcriptional factors involved in stress signal pathway. Such a protein array can contain antibodies against, e.g. c-Jun, ATF-2, Jun B and p53 etc. A protein array for ubiquitination can contain antibodies against cancer related proteins such as, e.g. p53, b-catenin and cyclin D1 etc.

Hydrophobic Group Addition

Addition of a hydrophobic group to a protein is another type of protein modification that affects the biological activity in a cell. Several hundreds of cellular and viral proteins are now known to be covalently modified by lipophilic moieties. These proteins include proteins, guanine nucleotide binding proteins, transmembrane receptors, and viral structural proteins. Among the most common hydrophobic modifications are fatty acids (myristate and palmitate), isoprenoids (farnesyl and geranylgeranyl), and glycosyl-phosphatidyl inositol anchors. The lipid attachment to these proteins influences protein-protein interactions, membrane binding affinity, and cellular signal transduction by the modified proteins. For example, a Src mutant devoid of myristoylation and a Ras mutant devoid of prenylation abrogate their cellular transformation ability. While most of lipophilic phosphorylation is irreversible, palmitoylation is a reversible process. Bradykinin treatment induces depalmitoylation of nitric oxide synthase and translocation of the depalmitoylated protein from membrane to cytosol. Lipophilic modification of proteins are often studied by metabolic labeling cells with a radioactive lipid subunit. Recently, lipid specific antibody such as anti-farnesyl antibody is used to identify the presence of lipophilic modification. A protein array for lipophilic modification can contain an array of antibodies each capable of capturing a protein of interest from a cell lysate for analyzing the presence of a specific lipid by a second antibody specific against the lipid of interest.

Another example of addition of a hydrophobic group is addition of an isoprenoid. For example, a protein array to detect such geranylgeranylation can be designed for analyzing a group of small guanine nucleotide binding proteins for the presence of genranylgeranylation. A geranylgeranylation protein array can contain antibodies (capture molecules) against Rap1A/Krev1, Rac, Ral and Rho etc. A protein array for farnesylation can be designed for analyzing a group of proteins for the presence of farnesylation. A farnesylation protein array can contain antibodies against H-Tas, N-Ras, K-Ras, Lamins A, Lamin B, transductin τ subunit, and Rhodopsin kinase.

Carbohydrate Addition

Addition of a carbohydrate to a polypeptide sequence of a protein can include, for example, glycosylation. Protein glycosylation is an important post-translational modification occurred in the lumen of the rough endoplasmic reticulum and in the Golgi. To date, there are estimates of over 200 glycosyltransferase enzymes involved in the addition of sugars onto newly synthesized proteins. Specific carbohydrate structures participate in cell-cell and cell-substratum interactions affecting processes such as lymphocyte trafficking, immune cell stimulation, embryogenesis, and cancer metastasis. Lectins as molecules can specifically recognize a certain structure of carbohydrate group attached to proteins. Antibodies against a specific carbohydrate group are also developed to detect the presence of the modified group on the protein of interest. For example, monoclonal antibody (Mab) 3E1.2 binds to multimers of the sialylated carbohydrate in a protein conformation-dependent manner on human mucins. The antigen for Mab 3E1.2 is elevated in breast cancer patients. Alteration of carbohydrate groups on proteins are found-to be associated with various cancers. Many of these alterations can be detected by lectins or carbohydrate-specific antibody. A protein array for glycosylation can contain an array of antibodies each capable of capturing a protein of interest from a sample of proteins for analyzing the presence of a specific carbohydrate group by a second carbohydrate-specific antibody (e.g. a polysaccharide-specific antibody) or a lectin specific for the particular carbohydrate group.

The present method can be used in different formats. For example, the target protein can be immobilized via the specific interaction between a capture molecule and the peptidic portion of the target protein, and then the protein modification status of the immobilized target protein is assessed. Alternatively, the target protein can be immobilized via the specific interaction between a capture molecule and the modification moiety or the combination of the modification moiety and the peptidic portion of the target protein, and then the identity of the of the immobilized target protein is assessed. Accordingly, in one specific embodiment, the target protein is first immobilized by a capture molecule that is capable of specifically binding to both a modified and an unmodified forms of a target protein, and then the modification status of the immobilized target protein is assessed by contacting the immobilized target protein with a detection molecule that is capable of specifically binding to the modified target protein but is not capable of specifically binding to the unmodified target protein itself. In this embodiment, the modification status of the immobilized target protein can also be determined by other suitable physical or chemical means (See e.g., Yan et al., J. Chromatogr. A., 808(1-2):23-41 (1998). For example, the physical or chemical means can comprise chemical or radioisotopic label of the protein modification moiety. Alternatively, the physical or chemical means can comprise any suitable analytical means, e.g., chromatographic, electrophoretic, protein sequencing, mass spectrometry and NMR means, for detecting the protein modification moiety. In another specific embodiment, the target protein is first immobilized by a capture molecule that is capable of specifically binding to a modified form of the target protein but is not capable of specifically binding to an unmodified form of the target protein, and then the identity of the immobilized target protein is assessed by contacting the immobilized target protein with a detection molecule that is capable of specifically binding to the unmodified target protein itself but is not capable of specifically binding to the modified target protein.

In a specific embodiment, two or more capture molecules are immobilized onto the solid support. A capture molecule can be, e.g. an antibody specific for a target protein, but may also be a non-antibody molecule, e.g. a lectin, or other protein, polypeptide, or peptide specific for a target molecule. The capture molecule may also be a non-protein molecule, for example a small molecule, nucleic acid, polynucleotide or other type of molecule capable of being immobilized onto the solid support and also capable of binding a target protein with some affinity and specificity. A solid support (e.g. a slide, wafer, membrane or filter) will have a variety of spots or positions on which populations of the same capture molecules can be placed. At each spot or location many molecules of a particular capture molecule can be immobilized. The amount of capture molecules required for detecting an amount of bound target protein will depend on the detection system being used (i.e. the more sensitive the detection system, the less capture molecules needed and the less capture molecule-target protein binding pairs will be generated and/or needed for detection), on the binding affinity between the capture molecule and the target protein, the expected relative amount of target protein in the protein sample, and other considerations. The amount of capture protein should be sufficient to generate detectable signal by the conventional means used in the laboratory. To date, the detection sensitivity for radioactive ³²P and fluorescence dye such as DBCI (a dicarbocyanine analog of indocyanine green) is 100,000 molecules. Therefore, the amount of the capture protein should be greater than 100,000 if the modification specific antibody is directly linked with a single radioactive 32P molecule or DBCI molecule (Silzel J, et al, 1998, Clinical Chemistry 44:2036-2043). However, less amount of the capture protein can be used if the modification specific antibody is linked with multiple detection molecules (32P, DBCI, etc.) through direct conjugation or enzyme amplification (see below for additional information). The size of the spot can be in the range of about 5 um to about 1 cm in diameter, for example. The amount of the capture protein “will depend on the size of the spot and the linear range of detection assay. Various means can be used to spot the capture molecules. For preparing a protein microarray (spot size is <1 mm in diameter), mechanical microspotting and ink jetting is preferred to be used (Shena M, et al, 1998, TIBTECH 16:302-306). For preparing protein array (spot size is >1 mm in diameter), spotting can be achieved through using conventional lab pipette.

The target molecule will preferably be bound to a capture molecule at an epitope or site of the target molecule that leaves any modification moiety on the target protein available for binding a detection molecule later. A capture molecule is selected for the specificity and affinity for binding particular target protein. The capture molecule can be immobilized on the solid support by following a procedure, for example, as follows: first blocking the protein array with blocking reagents (e.g. dry milk, gelatin or BSA containing solution) followed by rinsing away the blocking reagents using e.g., TBST or PBST. The protein array is then incubated with biologically activated sample such as cell lysate and tissue lysate etc. for a few hours. Proteinase inhibitors and phosphate inhibitors are usually included in the lysates. After the incubation, the protein array is then washed with TBST or PBST followed by incubation with modification-specific antibody for around 1 hour or so. The protein array is further washed with TBST or PBST and subjected to appropriate procedure for developing detection signals.

Once the capture molecules are immobilized on the solid support, the solid support is contacted with a biologically active sample of proteins comprising target proteins that may have undergone the subject protein modification. The target proteins specific for a given population of capture molecules bind the capture molecules and remain bound after washing. The detection can be achieved through linking modification-specific antibody with detection molecules such as fluorescent molecules or enzymes that are capable of depositing substrates such as fluorescent molecules, chromogenic substrates, and chemiluminecent substrates. Modification-specific antibody can also be linked to detection molecules indirectly through molecules such as biotin or other haptens such as fluorescein and digoxigenin etc. for amplification. Enzymes-linked strepavidin or enzyme linked-antibody against the hapten is then used for the detection. To achieve a greater amplification, the substrate for the enzymes can be linked to molecules such as biotin or other haptens such as fluorescein and digoxigenin etc., followed by detection by enzymes-linked strepavidin or enzyme linked-antibody against the hapten.

The bound target proteins are then contacted with detection molecules specific for the subject protein modification. All the different target proteins will be screened for the same protein modification. The detection molecule can be an antibody or other binding molecule specific for the modification being detected. The antibody can be a part of an antibody, for example, a polypeptide having specificity for the modification on the target proteins that are bound to capture molecules on the solid support. The detection molecules may be conjugated themselves to a detection means (e.g. enzymatic, fluorescent, chemiluminescent detection means), or may themselves need to contact a label or tag that is detectable. The tag, label, or detection means on the detection molecule can be, for example, a color tag, an oligo tag, a fluorescent tag, or a radio tag, etc. Where antibodies are used to detect the bound proteins, for example, a detection-ready antibody can be, e.g. conjugated with enzymes such as alkaline phosphatase (AP), horse radish peroxidase (HRP) or others for direct detection or conjugated with linker molecule such as biotin or other for subsequent linking to detection molecules.

For example, a modification specific antibody can bind an antigen captured on the solid support. From there a monoclonal antibody linked with a detection molecule or molecules binds the first antibody. Fluorescence molecules can then be detected without amplification of the signal. Alternatively, enzymes can be added that provide amplification of the signal, and then either fluorescent molecules, chromogenic substrates, or chemiluminescent substrates are detected. Another option for detection can be that a second monoclonal antibody binds the first monoclonal antibody, but this second monoclonal antibody is linked to indirect molecules such as, e.g. biotin, or other haptens such as fluorescein, or digoxigenin, etc. These indirect molecules react with a strepavidin or hapten antibody linked to enzymes. Detection then proceeds from either fluorescence molecules as a substrate, chromogenic molecules as a substrate, or chemiluminescent molecules as a substrate for the enzyme. Alternatively, for greater amplification, these enzymes can react upon substrates linked to another amplification molecule, e.g. biotin, fluorescein, or digioxigenin, etc., which in turn react with streptavidin or hapten antibody linked to enzymes that then react with fluorescent, chromogenic or chemiluminescent substrates. See Ausabel et al., eds., in the Current Protocol of Molecular Biology series of laboratory technique manuals. 1987-1997 Current-Protocols, 1994-1997 John Wiley and Sons, Inc.

In another specific embodiment, the organic phosphate can be converted into inorganic phosphate to detect inorganic phosphate in situ on the array (Kates, Techniques of Lipidology, 3^{rd} Ed. (North-Holland/Arnericam Elsevier, New York)). In still another specific embodiment, the phosphorylation substrates are left unlabeled on the array. The substrates are phosphorylated using adenosine 5′-O-(3-thiotriphosphate) (ATPγ-S) instead of ATP. After the kinase assay step, the thiophosphorylated product is then reacted with iodoacetyl derivative of a tag compound. The tag compound is either labeled with fluorecence, color or chemiluminecence that provide detection mean for the amount of phosphorylation (Jeong and Nikiforov, BioTechnique, 27:1232-1238 (1999)).

The protein modification status of a target protein, or a plurality of target proteins, in any sample can be assessed by the present method. Preferably, the sample to be assessed is a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

The protein modification status of any target protein, or any plurality of target proteins, can be assessed by the present method. Preferably, the target protein to be assessed is involved in a biological pathway, belongs to a group of proteins with identical or similar biological function, expressed in a stage of cell cycle, expressed in a cell type, expressed in a tissue type, expressed in an organ type, expressed in a developmental stage, a protein whose expression and/or activity is altered in a disease or disorder type or stage, or a protein whose expression and/or activity is altered by drug or other treatments.

In a specific embodiment, the present method can be used to assess protein modification profile of a particular tissue such as epithelium tissue, connective tissues, including blood, bone, and cartilage, muscle tissue and nerve tissue.

In another specific embodiment, the present method can be used to assess protein modification profile of a particular organ, i.e., any part of the body exercising a specific function, as of respiration, secretion or digestion.

In still another specific embodiment, the present method can be used to assess protein modification profile of a particular organism, such as plant, animal, bacteria, e.g., eubacteria and archaebacteria, virus, e.g., Classes I-VI viruses, or fungus.

In yet another specific embodiment, the present method can be used to assess protein modification profile of a particular disease or disorder”, such as infection, neoplasm (neoplasia), cancer, an immune system disease or disorder, a metabolism disease or disorder, a muscle and bone disease or disorder, a nervous system disease or disorder, a signal disease or disorder, or a transporter disease or disorder.

In yet another specific embodiment, the protein samples can come from tissues or cell lines, for example. The protein sample can be from, e.g. an animal, plant, fungus (e.g. yeast) or bacteria. The animal can be, e.g. a fish, amphibian, reptile, insect (e.g. drosophila), or mammal. The mammal can be e.g. a human, primate, dog, cat, rodent, goat, sheep, or cow.

Where a comparison is made using a protein array, e.g. a comparison between a control array and an array from a protein mixture of a particular condition or change in a condition, the control sample can be e.g. from a normal tissue and the experimental sample can be from changed tissue, e.g. diseased tissue, or the control and test sample can be from the same tissue or cells or from different animals; from the same tissues of a different developmental stage. The organisms in a comparison can be e.g. wild type, diseased, knockout or transgenic. The array could also represent different tissues or cells from the same body. The control sample can be, e.g. untreated cells and the experimental sample can be treated cells. The treatment can be various biological, physical or chemical treatments such as, for example, any drug treatment whether a known approved drug or a test drug. The treatment can also comprise such treatments, e.g. as administration of a growth factor treatment, UV irradiation, or other drugs, chemicals or therapies to treat a disease or condition or to cause a change in a condition.

The biologically active sample of proteins can be, for example contained in cell lysate. The proteins may be isolated from multiple cells, populations of different cells, tissue, serum, blood, body fluids, or other sources which potentially contain modulated proteins and which it is desirable to test for the protein modification that has occurred in the sample. Cell lysates may be prepared as described in Ausabel et al., eds., in the Current Protocol of Molecular Biology series of laboratory technique manuals. 1987-1997 Current Protocols, 1994-1997 John Wiley and Sons, Inc.

In another aspect, the present invention is directed to a method for identifying biologically distinguishable marker(s) associated with a biosample, which comprises: 1) assessing protein modification profile of a biosample through the above-described method; 2) assessing protein modification profile of a comparable control biosample through the above-described method; and 3) comparing the protein modification profile obtained in step 1) with the protein modification profile obtained in step 2) to identify biologically distinguishable protein modification profile marker(s) associated with said biosample. Preferably, the identified biologically distinguishable protein modification profile marker(s) are indicative of the protein modification profile of the biological source from which the biosample is derived.

C. Kits and Arrays for Detecting Protein Modification

In still another aspect, the present invention is directed to a kit for detecting protein modification, which kit comprises: a) a capture molecule immobilized on a solid support, said capture molecule is capable of specifically binding to a target protein; and b) means for assessing modification status and/or identity of said target protein.

Although the present kit can be used to assess the protein modification status of a single target protein at a time, the present kit is preferably used in a high-throughput format. For example, the kit can comprise a plurality of capture molecules that is immobilized on the solid support, each of said capture molecules is capable of specifically binding to a member protein of a group of structurally and/or functionally related target proteins.

The protein modification status (or profile) of any plurality, i.e., group, of target proteins can be assessed by the present kit. Preferably, the protein modification status (or profile) of a group of structurally and/or functionally related proteins is assessed.

Any molecule, or complex or combination therefor, that is capable of specifically binding to a target protein, or to one or more member(s) of a plurality of target proteins, can be used as the capture molecule in the present kit. In one specific embodiment, the capture molecule is capable of specifically binding to both a modified and an unmodified forms of a target protein. In another specific embodiment, the capture molecule is capable of specifically binding to a modified form of the target protein but is not capable of specifically binding to an unmodified form of the target protein. In a preferred embodiment, the capture molecule is an antibody, e.g., a polyclonal antibody, a monoclonal antibody, an antibody fragment retaining its desired binding specificity, or a combination thereof.

Any suitable solid support can be used in the present kit. In one example, the solid support can be a silicon, plastic, nylon, glass, ceramic, photoresist, rubber or polymer support. The solid support can be in any kind of suitable geometric forms, e.g., a flat support, a set of sticks, or a set of beads. Exemplary flat supports can comprise a slide, a chip, a filter, or a membrane.

Any protein modification, especially post-translational protein modification, can be assessed by the present kit. Exemplary protein modifications that can assessed by the present method include phosphorylation, acetylation, methylation, ADP-ribosylation, addition of a polypeptide side chain, addition of a hydrophobic group, and addition of a carbohydrate. In one specific embodiment, the phosphorylation to be assessed is phosphorylation on tyrosine, serine, threonine or histidine residue. In another specific embodiment, the addition of a polypeptide side chain to be assessed is the addition of ubiquitin. In still another specific embodiment, the addition of a hydrophobic group to be assessed is the addition of a fatty acid, e.g., myristate or palmitate, addition of an isoprenoid, e.g., farnesyl or genranylgenranyl, or addition of a glycosyl-phosphatidyl inositol anchor, e.g., a carbohydrate group comprises glycosyl.

The present kit can be used in different formats. For example, the target protein can be immobilized via the specific interaction between a capture molecule and the peptidic portion of the target protein, and then the protein modification status of the immobilized target protein is assessed. Alternatively, the target protein can be immobilized via the specific interaction between a capture molecule and the modification moiety or the combination of the modification moiety and the peptidic portion of the target protein, and then the identity of the of the immobilized target protein is assessed. Accordingly, in one specific embodiment, the target protein is first immobilized by a capture molecule that is capable of specifically binding to both a modified and an unmodified forms of a target protein, and then the modification status of the immobilized target protein is assessed by contacting the immobilized target protein with a detection molecule that is capable of specifically binding to the modified target protein but is not capable of specifically binding to the unmodified target protein itself. In this embodiment, the modification status of the immobilized target protein can also be determined by other suitable physical or chemical means. For example, the physical or chemical means can comprise chemical or radioisotopic label of the protein modification moiety. Alternatively, the physical or chemical means can comprise any suitable analytical means, e.g., chromatographic, electrophoretic, protein sequencing, mass spectrometry and NMR means, for detecting the protein modification moiety. In another specific embodiment, the target protein is first immobilized by a capture molecule that is capable of specifically binding to a modified form of the target protein but is nor capable of specifically binding to an unmodified form of the target protein, and then the identity of the immobilized target protein is assessed by contacting the immobilized target protein with a detection molecule that is capable of specifically binding to the unmodified target protein itself but is not capable of specifically binding to the modified target protein.

The protein modification status of a target protein, or a plurality of target proteins, in any sample can be assessed by the present kit. Preferably, the sample to be assessed is a biological sample.

The protein modification status of any target protein, or any plurality of target proteins, can be assessed by the present kit. Preferably, the target protein to be assessed is involved in a biological pathway, belongs to a group of proteins with identical or similar biological function, expressed in a stage of cell cycle, expressed in a cell type, expressed in a tissue type, expressed in an organ type, expressed in a developmental stage, a protein whose expression and/or activity is altered in a disease or disorder type or stage, or a protein whose expression and/or activity is altered by drug or other treatments.

In one specific embodiment, the kit can further comprise: a) instructions for using the kit; b) reagents and buffers; and/or c) a container(s) for the kit contents.

In yet another aspect, the present invention is directed to an array of protein capture molecules, which array comprises: a) a solid support; and b) a plurality of capture molecules immobilized on said solid support, wherein each of said molecules is capable of specifically binding to both a modified and an unmodified form of a member protein of a plurality of target proteins. Preferably, the plurality of target proteins comprises a group of structurally and/or functionally related proteins.

The modified and unmodified forms of the same target protein to be assessed by the present array can have same, but preferably, different biological activities. The modified and unmodified forms of the same target protein to be assessed by the present array can represent same, but preferably, different physiological conditions or biological statuses. The present array can be used to identify pathway activation. The present array can also be used to identify activation of a group of structurally and/or functionally related protein. The present array can further be used to generate a modification profile correlated to a physiological condition, drug treatment and disease. The present array can also be used to identify a physiological or pathological status. The present array can also be used to record biological perturbation caused by drug and other treatment.

In yet another aspect, the present invention is directed to an array of protein capture molecules, which array comprises: a) a solid support; and b) a plurality of capture molecules immobilized on said solid support, wherein each of said molecules is capable of binding to a specific epitope generated from modification of a modified protein, e.g., Rb.

In a specific embodiment, the solid supports can be a two-dimensional relatively flat surface. For example, the solid support can be a slide, a wafer, a filter, or a membrane. These supports can be made of any material to which the subject capture molecules can be immobilized, and upon which the protein modification may be detected with a detection molecule. Thus, for example, the solid supports can be made of glass, plastic, polymer, nylon, nitrocellulose, metal, or a favorable or useful mixture of any of these materials. For a wafer or rectangular two dimensional solid support, two or more populations of capture molecules are immobilized onto the solid support at different distinct locations in order to capture the target proteins and apply detection molecules to screen for protein modification in those captured target proteins.

The solid support may also be other than a rectangular two dimensional surface, and may be in the form of multiple sticks (e.g. flat sticks or strips, one each for a capture molecule population). For a particular protein modification, where two or more capture molecules are used, two or more sticks are made to capture and detect the particular protein modification sought. The solid support may also be collection of beads. A bead or group of beads will be coated with a particular capture molecule, and the entire population of beads will include beads specific for two or more target proteins having the same-type protein modification. Each bead will be coded or marked to be clear what target protein is captured on the beads, and thus to detect which protein is modulated. Both the bead and sticks may be made of any material suitable for the purpose described including, e.g. glass, plastic, polymer, nylon, nitrocellulose, metal, or a favorable or useful mixture of any of these materials. The beads, sticks, strips, or contiguous solid supports can be coded, e.g. with a bar code or ink mark to identify the support, and to perhaps designate an orientation, e.g. as with a contiguous solid support having a grid of capture molecules. In any event, the practitioner needs to keep track of what capture molecule is where on the contiguous solid support, stick, strip, or bead, so that the target protein is identifiable.

Two or more capture molecules are immobilized onto the solid support. A capture molecule can be, e.g. an antibody specific for a target protein, but may also be a non-antibody molecule, e.g. a lectin, or other protein, polypeptide, or peptide specific for a target molecule. The capture molecule may also be a non-protein molecule, for example a small molecule, nucleic acid, polynucleotide or other type of molecule capable of being immobilized onto the solid support and also capable of binding a target protein with some affinity and specificity. A solid support (e.g. a slide, wafer, membrane or filter) will have a variety of spots or positions on which populations of the same capture molecules can be placed. At each spot or location many molecules of a particular capture molecule can be immobilized. The amount of capture molecules required for detecting an amount of bound target protein will depend on the detection system being used (i.e. the more sensitive the detection system, the less capture molecules needed and the less capture molecule-target protein binding pairs will be generated and/or needed for detection), on the binding affinity between the capture molecule and the target protein, the expected relative amount of target protein in the protein sample, and other considerations. The amount of capture protein should be sufficient to generate detectable signal by the conventional means used in the laboratory. To date, the detection sensitivity for radioactive 32P and fluorescence dye such as DBCI (a dicarbocyanine analog of indocyanine green) is 100,000 molecules. Therefore, the amount of the capture protein should be greater than 100,000 if the modification specific antibody is directly linked with a single radioactive 32P molecule or DBCI molecule (Silzel J, et al, 1998, Clinical Chemistry 44:2036-2043). However, less amount of the capture protein can be used if the modification specific antibody is linked with multiple detection molecules (32P, DBCI, etc.) through direct conjugation or enzyme amplification (see below for additional information). The size of the spot can be in the range of about 5 um to about 1 cm in diameter, for example. The amount of the capture protein will depend on the size of the spot and the linear range of detection assay. Various means can be used to spot the capture molecules. For preparing a protein microarray (spot size is <1 mm in diameter), mechanical microspotting and ink jetting is preferred to be used (Shena M, et al, 1998, TIBTECH 16:302-306). For preparing protein array (spot size is >1 mm in diameter), spotting can be achieved through using conventional lab pipette.

The target molecule will preferably be bound to a capture molecule at an epitope or site of the target molecule that leaves any modification moiety on the target protein available for binding a detection molecule later. A capture molecule is selected for the specificity and affinity for binding particular target protein. The capture molecule can be immobilized on the solid support by following a procedure, for example, as follows: first blocking the protein array with blocking reagents (e.g. dry milk, gelatin or BSA containing solution) followed by rinsing away the blocking reagents using e.g., TBST or PBST. The protein array is then incubated with biologically activated sample such as cell lysate and tissue lysate etc. for a few hours. Proteinase inhibitors and phosphate inhibitors are usually included in the lysates. After the incubation, the protein array is then washed with TBST or PBST followed by incubation with modification-specific antibody for around 1 hour or so. The protein array is further washed with TBST or PBST and subjected to appropriate procedure for developing detection signals.

In another specific embodiment, an array of immobilized antibodies are used in the kits for detecting protein modification. Any antibodies, whether polyclonal, monoclonal, single chain, Fc fragment, Fab fragment, F(ab)₂ fragment, or a mixture thereof, can be used to produce the antibody arrays.

The antibody array used in the kit can be produced on any suitable solid surface, including silicon, plastic, glass, polymer, such as cellulose, polyacrylamide, nylon, polystyrene, polyvinyl chloride or polypropylene, ceramic, photoresist or rubber surface. Preferably, the silicon surface is a silicon dioxide or a silicon nitride surface. Also preferably, the array is made in a chip format. The solid surfaces may be in the form of tubes, beads, discs, silicon chips, microplates, polyvinylidene difluoride (PVDF) membrane, nitrocellulose membrane, nylon membrane, other purous membrane, non-porous membrane, e.g., plastic, polymer, perspex, silicon, amongst others, a plurality of polymeric pins, or a plurality of microtitre wells, or any other surface suitable for immobilizing proteins and/or conducting an immunoassay.

The antibodies can be attached to the solid surface by any methods known in the art (see generally, WO 99/39210, WO 99/40434). For example, the antibodies can be attached directly or through linker(s) to the surface. The antibodies can be attached to the surface through non-specific, specific, covalent, non-covalent, cleavable or non-cleavable linkage(s). The cleavable linkage can be cleavable upon physical, chemical or enzymatic treatment. The arrays can be arranged in any desired shapes such as linear, circular, etc.

In one example, antibody array can be printed on a solid surface using pins (passive pins, quill pins, and the like) or spotting with individual drops of solution (WO 99/40434). Passive pins draw up enough sample to dispense a single spot. Quill pins draw up enough liquid to dispense multiple spots. Bubble printers use a loop to capture a small volume which is dispensed by pushing a rod through the loop. Microdispensing uses a syringe mechanism to deliver multiple spots of a fixed volume. In addition, solid supports, can be arrayed using piezoelectric (ink jet) technology, which actively transfers samples to a solid support. In addition, the methods disclosed in WO 95/35505 can also be used. The method and apparatus described in WO 95/35505 can create an array of up to six hundred spots per square centimeter on a glass slide using a volume of 0.01 to 100 nl per spot. Suitable concentrations of antibody range from about 1 ng/μl to about 1 μg/l. Further, other methods of creating arrays, including photolithographic printing (Pease, et a., PNAS 91(11):5022-5026, 1994) and in situ synthesis can be used.

Methods for covalent attachment of antibodies to a solid support are known in the art. Examples of such methods are found in Bhatia, et al., Anal. Biochem. 178(2):408-413, 1989; Ahluwalia, et al., Biosens. Bioelectron. 7(3):207-214, 1992; Jonsson, et al., Biochem. J. 227(2):373-378, 1985; and Freij-Larsson, et al., Biomaterials 17(22):2199-2207, 1996, all of which are incorporated by reference herein in their entirety

Methods of reducing non-specific binding to a solid surface are well known in the art and include washing the arrayed solid surface with bovine serum albumin (BSA), reconstituted non-fat milk, salmon sperm DNA, porcine heparin, and the like (see Ausubel, et al., Short Protocols in Molecular Biology, 3rd ed. 1995).

D. Array, Kits and Methods for Detecting Enzymatic Activity

In yet another aspect, the present invention is directed to an array of enzyme substrates, which array comprises: a) a solid support; and b) a plurality of substrates immobilized on said solid support, wherein each of said substrates is a substrate of a member enzyme of a group of structurally and/or functionally related enzymes. Preferably, at least one of the member enzymes catalyzes a protein modification reaction.

In yet another aspect, the present invention is directed to a kit for detecting enzymatic activity, which kit comprises: a) an array comprising a solid support, and a plurality of substrates immobilized on said solid support, wherein each of said substrates is a substrate of a member enzyme of a group of structurally and/or functionally related enzymes; and b) means for assessing activity of each of the member enzymes.

In yet another aspect, the present invention is directed to a method for detecting enzymatic activity in a sample, which method comprises: a) contacting a sample containing or suspected of containing a group of structurally and/or functionally related target enzymes with a plurality of substrates immobilized on a solid support, wherein each of said substrates is a substrate of a member enzyme of said group of target enzymes under conditions suitable for said target enzymes to catalyze enzymatic reactions involving said immobilized substrates; and b) assessing enzymatic activities of said target enzymes. Preferably, at least one of the target enzymes catalyzes a protein modification reaction.

The invention also provides a method of detecting enzymatic activity in a biologically active or activated (e.g. also an enzymatically active or activated) sample of proteins. The method is practiced by providing a solid support comprising two or more enzyme substrate molecules immobilized on the support each of which can act as a substrate for an enzyme capable of the same enzymatic modulation on a target substrate; contacting the solid support with a biologically active sample comprising enzymatically active proteins that may act on the immobilized substrates under enzymatic conditions and perform a detectable enzymatic reaction on the immobilized substrate; and detecting an enzyme modulation on the substrate with a detection mean (such as 32P for phosphorylation) or molecule that specifically binds the subject enzymatic modulation in order to detect whether or not the sample of proteins comprise a certain enzymatic activity, wherein the presence of a particular enzymatic activity in a sample imparts information about biological activity present in the sample. The enzymatic activity can comprise an enzymatic activity selected from the group consisting of kinase, phosphatase, transferase, lipid kinase, isomerase, glycosidase, lipase, ligase, nuclease, peptidase, protease, ubiquinase, glycosyltransferase and glycosylase.

The invention also includes a kit for performing the detection of enzymatic activity in a biologically active or activated sample of proteins. The kit comprises a solid support of two or more enzyme substrate molecules immobilized on the support, and reagents for practicing the method just described. The invention also provides a solid support comprising two or more enzyme substrate molecules immobilized on the support, each of which can act as a substrate for an enzyme capable of the same enzymatic modulation on a target substrate; wherein the solid support provides an environment for an enzymatic reaction at the immobilized substrates under enzymatic conditions, and further wherein any modulation of the substrate by the enzyme can be detected with a detection molecule under detection conditions on the solid support.

E. Exemplary Methods, Kits Arrays and Uses Thereof

The following describes certain exemplary or preferred methods, kits, arrays and their uses thereof.

To prepare a protein array on a two-dimensional surface, capturing molecules at appropriate concentration are laid on a specific location of the surface for a few hours or overnight for absorption to the surface. The surface for absorption should possess high protein-binding capability if the capturing molecule is a protein. These surfaces include coated slides, nitrocellulose or nylon membranes are commercially available: nitrocellulose or nylon membrane and membrane slides can be from Schleicher & Schuell (Keene, N.H.); glass slides can be purchased from Xenopore (Hawthorne, N.J.).

For nitrocellulose and nylon membrane, for example, capturing molecules can be coated by forced absorption onto the membranes through vacuum. Bovine serum albumin (BSA) or milk solution is then used to saturate the nonspecific binding of the membrane to derive a prepared protein array. For glass slides the antibody to be bound is simply dissolved in a buffer solution, e.g. as described in the instructions from Xenopore (Hawthorne, N.J.). At pH above the isoelectric point of the protein (important because the binding takes place through the amine group on the protein, and these must be in the free form for binding in the well) is incubated for 2-3 hours at 37 degrees C., using 50 mM Na₂ CO₃/NaHCO₃ solution of pH 9.6. This results in the formation of a covalent bound between the surface and the protein. The antibody solution is then incubated. Antibody cal also be coated onto strepavidin coated slide through biotin-conjugation.

To practice the invention, cell lysate can be prepared from the source that needs to be studied. The source can be a cell line, tissue or animal. Various methods have been used extensively for the preparation of cell lysate. The cell lysate is then used to incubate with a protein array. The protein array is a two-dimensional surface or a population of beads or a population of sticks. Each protein array could contain an array of antibodies each with multiple copies spotted on a specific area of the two-dimensional surface or a single type of bead or stick. After the incubation of cell lysate with a protein array followed by a few washing steps, proteins recognizing the antibodies on the protein array are bound on the array. The bound protein array is then stained with modification group specific antibodies (second antibodies). The second antibody is preferred to be pre-linked with a detection molecule such as a biotin, an enzyme, a dye or a radioactive tag that becomes visible recognized by naked eyes after development. The amount of the protein, modification of the protein and conformation change of the protein is determined through the detection system built on the second antibody. For the protein array made on a two-dimensional surface, the location of capturing antibody immobilized on the array serves as a reference for the identity of detected proteins. For the protein array made on a population of beads or sticks, a tag on each kind of bead or stick serves as a reference for the identity of detected proteins. The tag can be color, fluorescence, oligo, radiofrequency tag and other tag that can be easily used to separate beads with different tags. Protein array could also contain a population of enzyme substrates that are used to determine activity of multiple enzymes simultaneously, as described below.

The prepared protein array is incubated with a cell lysate where proteins of interest are present for a period of time. The incubation allows capture of proteins of interest onto the protein array. The protein array is then washed several times by appropriate solution (a washing solution can comprise TBS (50 mM Tris, 125 mM NaCl, pH 7.4) and PBS (50 mM NaPO₃, 125 mM NaCl) with addition of detergent such as Tween-20 to become TBST and PBST) a few times followed by incubating with a second antibody for a period of time. After second antibody incubation, the protein array is then washed a few times again with TBST to get rid of nonspecific binding and develop chemiluminescence signal or colormetric signal according to the detection system conjugated to the second antibodies. If the second antibody is directly conjugated by horseradish peroxidase (HRP) or alkaline phosphatase (AP) enzymes, colormetric substrates can be used to develop signal. If the second antibody is conjugated by biotin, for example, a chemiluminescence detection system (Vector Laboratories, Burlingame, Calif.) can be applied as well.

The methods, kits, and compositions of the invention can be used, e.g. to monitor modification of group of proteins of interest, by identifying affected proteins in, for example, knockouts, transgenic animals, or microbile-infected or diseased model animals. The invention can also be used to determine molecular mechanisms of pathology in disease or tumor biology, to discover the effect of therapeutic drugs or environmental toxins on critical pathways, to understand the roles of critical genes in, for example, cardiovascular and neurological disease, cancer, toxicology, cell-cycle regulation, apoptosis, and stress response, as a molecular diagnostic for identifying defective proteins, and to classify disease type and disease stage in clinical samples.

Monitoring protein modification using protein arrays can be accomplished in the context of, for example, a group of receptor tyrosine kinases (RTK). Some tumor formations are associated with activation of RTKs, such as EGFR family receptors). Tyrosine kinase receptors can play a pivotal role in the formation, growth and metastasis of human cancers. To date, more than thirty tyrosine kinase receptors have been found belonging to six families including EGF family, FGF family, PDGF family, insulin family, NGF family and HGF family. Among these receptors, EGF family receptors, FGF family receptors, PDGF receptors in PDGF receptor family, IGF receptors in insulin receptor family and HGF receptor/Met in HGF receptor family have been found to be involved in the tumor formation. VEGF receptors in PDGF receptor family and FGF receptors are involved in angiogenesis to support the growth of tumor and the formation of metastasis. The signaling of HGF receptor/Met from HGF receptor family also induce the invasiveness and metastatic potential of various cell types. An RTK protein array provides an efficient way for screening abnormally activated RTK in tumor tissues. For RTK protein array, antibodies against RTKs are spotted onto a surface and their activation is detected by anti-phosphotyrosine antibody. Examples of arrays along these lines can include, e.g. EGFR family protein array (EGFR, ErbB-2, ErbB-3 and ErbB-4); angiogenic RTK protein array (FGFR1, FGFR2, FGFR3, FGFR4, FLT1, Flk1/KDR, FLT4); mitogenic RTK (EGFR, ErbB-2, ErbB-3 and ErbB-4, IGFR, PDGFR, FGFR1, FGFR2, FGFR3, FGFR4, FLT1, Flk2/Flt3, Flk1/KDR, FLT4).

More specifically, a protein array for EGF family receptors (also called the ErbB family of receptors) can be organized along the following known biology of that receptor family. There are four members: EGFR, ErbB-2, ErbB-3 and ErbB4. EGFR and ErbB-2/neu are prototypes for a family of structurally related transmembrane proteins that play a role in the development and progression of cancer. Like other tyrosine kinase receptors, ErbB family receptors are activated through tyrosine phosphorylation upon dimerization induced by their ligand binding. The solid support can be contacted with a cell lysates from, for example, breast cancer cell lines to determine the identity and levels of activated EFG receptors in the family that are present in any particular lysate.

A group of proteins involved in a signal transduction pathway can be monitored using a protein array. For example see Table I for p44/42 MAP kinase, p38 MAP kinase, JNK and Akt signal pathways). The phosphorylation of proteins are detected by, e.g. phosphotyrosine-specific antibody, phosphoserine-specific antibody, phosphoserine-specific antibody, and phospho-threonine-proline antibody, or also for example, an antibody specific to a phosphorylated residue of a protein such as phosphorylated c-Jun at Ser 73.

A group of proteins involved in a common biological function may also be monitored using a protein array. For example, the Stat family of proteins can be monitored. Stat (signal transducers and activators of transcription) are a class of transcription factors that transmit signals for various cytokine and growth factors from cytoplasm to nucleus. Stats are activated through phosphorylation. There are 7 Stat family members to date. Stat 1 transmits signal for IFN α/β, IFN α and others; Stat 2 for IFN α/β; Stat 3 for gp130 users and others; Stat 4 for IL-12, IFN α/β; Stat 5a and Stat 5b for PRL, GH, EPO and γc users; Stat 6 for IL-4 and IL13. In addition, some Stats are also activated by other mitogenic signal and stress signal. For example, Stat 3 is activated through p44/42 MAP kinase signaling pathway and JNK signaling pathway. Stat1 can be also activated through JNK signaling pathway and was found to play an important role in inducing and maintaining constitutive levels of Caspases for apoptosis. A Stat family protein array can contain antibodies against each Stat and the activation of Stat can be detected by phosphorylation specific antibodies, such as those described herein.

A group of proteins whose modification is associated with a certain condition of a type of cell or tissue can be monitored using a protein array. The condition can be, for example, ischemia. Ischemia is a medical condition that induces many cellular response in brain. Many key regulators of various signaling pathways have been activated by phosphorylation. They can include CREB, ATF2, c-Jun, Jun B, Jun D, c-Fos, Rb, p44/42 MAP kinase, p38 MAP kinase, JNKs. Ischemia condition protein array can include proteins whose modification such as phosphorylation is associated with Ischemia condition. This protein array can be used for classifying the degree and type of Ischemia condition through generating a fingerprint of phosphorylation status of those Ischemia related proteins.

An array can be used to detect and/or monitor a group of marker proteins whose modification is associated with a certain condition such as a disease condition. For example a protein array for typing human lung cancer can be constructed. Elevated tyrosine phosphorylation of EGF receptor (EGF-r)(p 185), Erb B-2, beta-catenin and p125FAK have been found to be associated with human lung cancer. The elevation of tyrosine phosphorylation of p125FAK is restricted to cancerous lung tissues and is closely correlated with the nodal involvement of cancer and disease-free survival time. A protein array for typing human lung cancer can include antibodies against these four proteins and their tyrosine phosphorylation status is determined by using anti-phosohotyrosine antibody. This protein array can be used to classify different stage of lung cancers into various category.

Other uses of the protein array methods, kits and compositions can include identification of signal pathways. Pathways affected in, for example, knockouts, transgenics, microbile infected animals, or disease model animals can be studied. Thus, an array can be used to determine molecular mechanisms of pathology in disease and/or tumor biology, for example; to discover the effect of therapeutic drugs or environmental toxins on critical pathways; to understand the roles of critical pathways in cardiovascular and neurological disease, cancer, toxicology, cell-cycle regulation, apoptosis, and stress response. It can also serve as molecular diagnostic mean for identifying defected pathways.

A family of proteins possessing a similar biochemistry property but involving different biological pathways can be studied and/or monitored using a protein array. For example, an MKK (MEK) family protein array can be made. Mitogen-activated protein kinases (MAPKs) mediate many of the cellular effects of growth factors, cytokines and stress stimuli. Their activation requires the phosphorylation of a threonine and a tyrosine residue located in a Thr-X-Tyr motif (where X is any amino acid) (Lawler S, et al. Current Biology 1998, 8:1387-1390). This activation is carried out by a family of enzymes known as MAP kinase (MKKs or MEKs). To date, there are seven MKKs identified. These MKKs were found to activate different MAPKs that involve in p44/p42 MAP kinase (Erk1/2) pathway, SAPK/JNK pathway and p38 MAP kinase pathway, etc. MKK1 and MKK2 activate p⁴4/p42 MAP kinase in p44/p42 MAP kinase (Erk1/2) pathway, MKK3, MKK4 and MKK6 activate p38 MAP kinase in p38 MAP kinase pathway, MKK4 and MKK7 activate JNK in SAPK/JNK pathway and MKK5 activates ERK5/BMK1 whose biological pathway is await to be discovered. An MKK family protein array can consist of most or all MKK family members. This protein array can allow simultaneous identification of activated MKKs (in their phosphorylated form) in a single assay. The activated MKKs indicate activation of their corresponding signal pathways. Therefore, MKK family protein array can serve as an efficient way for identifying activated signal pathways.

Similarly, a MAP kinase family protein array can be made. In mammalian systems, five distinguishable MAP kinase (MAPK) have been identified. These MAPKs are involved in the extracellular signal-regulated kinase 1 and 2 (ERK1/2) cascade, which preferentially regulates cell growth and differentiation, as well as the c-Jun N-terminal kinase (JNK) and p38 MAPK cascades, which function mainly in stress responses like inflammation and apoptosis. The last member ERK5 is activated by MKK5 and is thought to transmit signal for proliferation. MAPK family protein array can consist of most or all MAPK family members. This protein array allows simultaneous identification of activated MAPKs (in their phosphorylated form) in a single assay. The activated MAPKs indicate activation of their corresponding signal pathways. Therefore, MAPK family protein array can serve as an efficient way for identifying activated signal pathways.

Groups of proteins whose modification signals activation of a particular signal pathway can be studied and/or monitored using a protein array, which can be called a pathwayfinder protein array. The following two lists identify the pathway on the left and the corresponding marker proteins on the right that would be captured on the protein array and detected for their corresponding modification.

TABLE 1
Exemplary marker proteins for certain pathways

| Pathway | Marker protein |
|---|---|
| P44/42 MAP kinase pathway | phospho-MAPK 1 & 2 |
| | phospho MKK 1 & 2 |
| P38 MAP kinase pathway | phospho MKK 4 & 7 |
| | phospho-JNK |
| | phospho-c-Jun |
| JNK pathway | phospho p38 MAPK |
| | phospho MKK3 & 6 |
| NFkB pathway | phospho-ikB |
| CREB pathway | phospho-CREB |
| P70 S6 kinase pathway | phospho-p70 S6 kinase |
| | phospho-S6 |
| PI-3 kinase/Akt pathway | phospho-Akt |
| JAK/Stat pathway | phospho-JAK or phospho-Stat |
| TGFb/Activin pathway | phospho-Smad 2 & 3 |
| BMP 2 & 4 pathway | phospho-Smad 1 & 5 |
| BMP7 pathway | phospho-Smad 5 |
| NFAT pathway | dephosphorylated-NFAT1 |
| Wnt pathway | phospho-GSK3 |
| P53 pathway | phospho-p53 |
| Insulin signaling | phospho-GSK3 |

A pathwayfinder protein array could contain antibodies (capture molecules) against some of marker proteins listed above. After reacting with a protein sample, the capture molecules would capture the corresponding marker proteins, and the phosphorylation state of these marker proteins would be detected by an appropriate phosphorylation-specific antibody.

A protein array can be used to monitor enzymatic activities of a group of proteins of interest. For example a group of specific kinases can be studied for their activities, listed in Table 1, and also elsewhere herein. Also a group of proteases can be studied for their, activities, for example, a caspase family substrate array can be made. Caspases are involved in apoptosis. There are a total of 10 caspases identified to date. Caspase family substrate array can allow simultaneous analyzing of several or all caspases for their activities in a single assay. In caspase family substrate array, the substrate for each caspase is immobilized on a solid surface. These substrates are usually in colormetric or fluorometric format and can be detected after the cleavage. For example, a fluorescent or a colormetric dye is attached to the substrate molecules such as DEVD as Caspase-3 substrate or IETD for Caspase-8 substrate. Upon cleavage of the substrate by caspase, the fluorescent or color signal is decreased proportionally to the activity of corresponding caspase. Alternatively, two dye molecules that quench their fluorescent signal can be attached to a single substrate. After the cleavage by the caspase resulting a loss of a fluorescent dye, a fluorescent signal is generated from the residual substrate.

Various activation states of a protein can be monitored using a protein array. For example, an Rb phosphorylation site protein array can identify important activation states of retinoblastoma (Rb). The retinoblastoma tumor suppressor, Rb, regulates cell proliferation by controlling progression through the restriction point within the GI phase of the cell cycle. Rb has three functionally distinct binding domains and interacts with critical regulatory proteins including the E2F family of transcription factors, c-abl tyrosine kinase and proteins with a conserved LXCXE motif. Cell cycle-dependent phosphorylation by cdks controls Rb activity by preventing binding to these regulatory targets. Rb can be phosphorylated at a multiplicity of sites and differential phosphorylation has been shown to modulate Rb function both in vitro and in vivo. Rb phosphorylation site protein array contains a group of phospho-Rb antibodies, each against a specific form of phospho-Rb. These antibodies can include anti-phospho-Rb (Ser795), anti-phospho-Rb (Ser249/252), anti-phospho-Rb (Thr373), anti-phospho-Rb (Ser780), anti-phospho-Rb (Ser8O7/811). After capturing various form of Rb on the protein array, a Rb antibody recognizing both unphosphorylated or phosphorylated Rb is used to determined the amount of Rb at each conformation.

Kits of the invention are designed to detect protein modification in a biologically active sample of proteins. The kits comprise a solid support of 2 or more capture molecules immobilized on the solid support, each of which can specifically bind a target protein-that is capable of a subject protein modification; a detection molecule specific for the subject protein modification in order to detect whether or not a captured target protein comprises the subject protein modification, wherein a target protein comprising the subject protein modification imparts information about biological activity present in the sample; and instructions for use of the kit. The instructions may follow many of the guidelines set forth above for practicing the method of the invention. The kit components are as described above for the solid supports and detection molecules. Other reagents, tools and/or buffers may also be included in the kits. The kits may also comprise containers for the kit contents.

The invention comprises also a composition comprising a solid support comprising 2 or more capture molecules immobilized on the support each of which can specifically bind a target protein that is capable of protein modification; wherein target proteins that specifically bind the capture molecules will form binding pairs on the support; and a bound target protein on the support can be detected with a detection molecule specific for protein modification; further wherein if a target protein comprises protein modification, information about biological activity present in a sample that comprised the target proteins is imparted. The solid supports are constructed and prepared essentially as described above and elsewhere herein. The subject protein modification on a solid support can comprises a modification selected from the group consisting of phosphorylation, acetylation, methylation, ADP-ribosylation, addition of a polypeptide side chain, addition of a hydrophobic group, and addition of a carbohydrate. The subject protein modification on the solid support can be a phosphorylation and the phosphorylation can comprises tyrosine, serine or threonine phosphorylation. The subject protein modification can be is addition of a polypeptide side chain, and the polypeptide side chain can be, for example, ubiquitin. The subject protein modification can be addition of a hydophobic group and the hydrophobic group can comprises a hydrophobic moiety selected from the group consisting of a fatty acid, an isoprenoid, and a glycosyl-phosphatidyl inositol anchor. The fatty acid can be myristate or palmitate. The isoprenoid can be farnesyl or genranylgenranyl. The carbohydrate addition can comprise a glycosylation. The capture molecules on the solid support can be antibodies. The detection molecules can be antibodies or lectins. The solid support can comprise any types-of solid support described above. The biological activity present in the sample can comprise any biological activity that is implicated by a protein modification, including any described herein.

Applying the principles of the basic method of the invention, the invention also provides a method of identifying and characterizing a changed condition. The changed condition is capable of manifestation e.g. by protein modification or enzymatic activity. The method is practiced by contacting two solid supports each comprising 2 or more capture molecules immobilized on the support with a first and second sample comprising target proteins; wherein each solid support comprises an identical amount and pattern of capture molecules and each capture molecule on each solid support can specifically bind a target protein that is capable of a subject protein modification; and further wherein the first sample represents an unchanged condition and the second sample represents a changed condition; contacting the first and second solid support with detection molecules capable of detecting modulated target proteins bound to capture molecules on the solid support; and comparing the detected modifications on the first and second solid supports to identify and characterize the changed condition. The changed condition can be a change in any condition biologically possible provided the change in the condition may be detected by the presence or absence of a protein modification or enzymatic activity present in the test protein sample in which the condition may have occurred or is occurring. Thus, the changed condition may comprise, for example, a condition selected from the group consisting of a disease, a drug treatment, a chemical treatment, a test drug effect, a physical change, a biological change, a developmental stage, a disease stage, and a disease progression.

Exemplary protein arrays may be described, but the following exemplary arrays and those depicted in the subsequent tables are not limiting of the invention, and by no means provide an exhaustive review of all types of protein arrays or solid substrates or subject protein modifications or subject enzymatic activity possibly useful along the principles of the invention. A protein array may comprising immobilized capture molecules on a solid support capable of specifically binding certain proteins, wherein the capture molecules are specific for 2 or more proteins selected from the group consisting of Rac, MEKK3, MEK4, MEK7, JNK1, JNK2, c-jun, Elk-1, Jun D, and ATF-4. A protein array may comprise immobilized capture molecules on a solid support capable of specifically binding certain proteins, wherein the capture molecules are specific for a 2 or more of proteins consisting of mitogenic pathway group comprising phosphorylation of any of p^{44/42} MAP Kinase (Thr202/Tyr204) and MEK1/2 (Ser217/221), a stress pathway group comprising phosphorylation of any of SAPK/JNK (Thr183/Tyr185), p38 MAP kinase (Thr180/Tyr182), MKK3/MKK6 (Ser189/207), and SEK1/MKK4 (Thr223), a cell survival pathway group comprising phosphorylation of any of Akt (Ser473), Bad (Serll2/136) and p70 S6Kinase (Ser411, Thr421/Ser424), activation of NFkB signal pathway comprising phosphorylation of ikB, activation of mitogenic/differentiation signal pathway comprising phosphorylation of any of ELK1, CREB, Etsl, Ets2, CBP, PEA3, p90^{rsk} and CEBP, activation of cytoskeletal organization signal pathway comprising phosphorylation of any of c-Jun, Elk1, ATF2, c-myc, SAPla and PEA3, and apoptosis/stress signal pathway comprising phosphorylation of any of ATF1, ATF2, Elk1, Max, CHOP, CREB, SAP1a and MAPKAPK-2. A protein array may comprising immobilized capture molecules on a solid support capable of specifically binding certain proteins, wherein the capture molecules are specific for 2 or more proteins selected from the group consisting of phosphorylated proteins of the ErbB family receptors comprising EGFR, ErbB-2, ErbB-3 and ErbB-4. A protein array may comprise immobilized capture molecules on a solid support capable of specifically binding certain proteins, wherein the capture molecules are specific for 2 or more phosphorylated proteins selected from the group consisting of EGF receptor, PDGF receptor, SOS, Src, and p44/42 MAP Kinase. A protein array may comprise immobilized capture molecules on a solid support capable of specifically binding certain proteins, wherein the capture molecules are specific for 2 or more phosphorylated proteins selected from the group consisting of p44/42 MAP Kinase, MEK1/2, SAPK/JNK, p38 MAP kinase, MKK3/MKK6, SEK1/MKK4, Akt, Bad and p70 S6 Kinase. A protein array may comprise immobilized capture molecules on a solid support capable of specifically binding certain proteins, wherein the capture molecules are specific for 2 or more proteins selected from the group consisting of c-Jun, ATF-2, Jun B, p53, b-catenin and cyclin D1. A protein array may comprise immobilized capture molecules on a solid support capable of specifically binding certain proteins, wherein the capture molecules are specific for 2 or more genranylgenranylated proteins selected from the group consisting of Rap1A/Krev1, Rac, Ral and Rho. A protein array comprising immobilized capture molecules on a solid support capable of specifically binding certain proteins, wherein the capture molecules are specific for 2 or more farnesylated proteins selected from the group consisting of H-Tas, N-Ras, K-Ras, Lamins A, Lamin B, transductin T subunit, and Rhodopsin kinase. A protein array comprising immobilized capture molecules on a solid support capable of specifically binding proteins active in a function selected from the group consisting of mitogenesis, insulin activation or deactivation, apoptosis, cell survival, stress signaling, geranylation, farnesylation, tyrosine phosphorylation, serine phosphorylation, threonine phosphorylation, kinase activity, NFKB activation, JAK/STAT signaling, ubiquitination, proteins having lipid moieties, protein kinase C signaling, cell adhesion, cytoskeletal organization, and receptor signaling.

Exemplary enzyme substrate arrays can include, for example, the peptide sequence EAIYAAPFAKKK as a peptide substrate for Abl protein tyrosine kinase. Another substrate: KRQQSFDLF can be a peptide substrate for calmodulin-dependent protein kinase. Protein substrates can include, for example, ATF-2 as a substrate for p38 kinase and SAPK/JNK. c-Jun can be a substrate for SAPK/JNK kinase. Elk1 can be a protein substrate for MAPK/ERK and SAPK/JNK. Inactive p⁴² MAP kinase can be a protein substrate for MEK1 and/or MEK2 kinases. MBP can be a protein substrate for c-Raf kinase. With some of these protein substrates, the cite of activity (e.g. the peptide sequence) may suffice as a substrate for detecting the kinase activity. Thus, for example, a protein array for kinase enzymatic activity could include, e.g. ATF-2, c-Jun, Elk1, inactive p42 MAP kinase and MBP as substrates for the respective above identified kinase enzymes.

TABLE 2
Exemplary Target Proteins for Various Protein Arrays

| Far- | gen- | | p38 MAP | | p44/p21 | | Kinases |
|---|---|---|---|---|---|---|---|
| nesyla- | genra- | JNK | kinase | Ubiquit- | MAP | | (enzynme |
| tion | nylation | stress | Stress | ination | kinase | AKT | substrate) |
| H- | Rap1A | MKK4 | p38 | c-Jun | p44 MAP | Akt | ATF-2 |
| Tas | | MAPK | | kinase | Ser473 | substrate | |
| | | | Thr180 | | Thr202 | Thr308 | for p38 |
| | | | Tyr182 | | Tyr204 | | kinase |
| | | | | | | | and |
| | | | | | | | SAPK/JNK |
| N- | Krev1 | MKK7 | MKK3 | ATF-2 | p42 MAP | Bad | c-Jun |
| Ras | | | Ser189 | | kinase | Ser112 | substrate |
| | | | Ser207 | | Thr202 | Ser136 | for |
| | | | | | Tyr204 | SAPK/JNK | |
| | | | | | | | kinase |
| K- | Rac | JNK1 | MKK6 | Jun B | MEK1 | p70- | Elk1 |
| Ras | | | Ser189 | | Ser217 | S6 | substrate |
| | | | Ser207 | | Ser221 | kinase | for |
| | | | | | | Ser411 | MAPK/ERK |
| | | | | | | Thr421 | and |
| | | | | | | | SAPK/JNK |
| | | | | | | Ser424 | |
| Lamins | Ral | JNK2 | SEK1 | p53 | MEK2 | GSK3 | inactive |
| A | | | MKK4 | | Ser217 | | p42 MAP |
| | | | Thr223 | | Ser221 | | kinase |
| | | | | | | | substrate |
| | | | | | | | for |
| | | | | | | | MEK½ |
| | | | | | | | kinases |
| Lamin | Rho | c-jun | ATF1 | b- | ELK1 | | MBP |
| B | | | | catenin | Ser383 | | substrate |
| | | | | | Ser389 | | for c-Raf |
| | | | | | | | kinase |
| trans- | | ELK1 | ATF2 | cyclin | Stat3 | | |
| ductin | | | | D1 | Ser727 | | |
| τ | | | | | | | |
| subu- | | | | | | | |
| nit | | | | | | | |
| Rho- | | ATF2 | ELK1 | | Etsl | | |
| dopsin | | | Ser383/389 | | | | |
| kinase | | | | | | | |
| | | c-myc | MAX | | SAP1a | | |
| | | | Ser62 | | | | |
| | | SAP1a | CHOP | | CREB | | |
| | | PEA3 | CREB | | CBP | | |
| | | | Sen133 | | | | |
| | | | SAP1a | | PEA3 | | |
| | | | c-myc | | CEBP | | |
| | | | Ser62 | | | | |

The following example is included for illustrative purposes only and is not intended to limit the scope of the invention.

### F. EXAMPLES

1. Preparation of a Membrane Protein Array

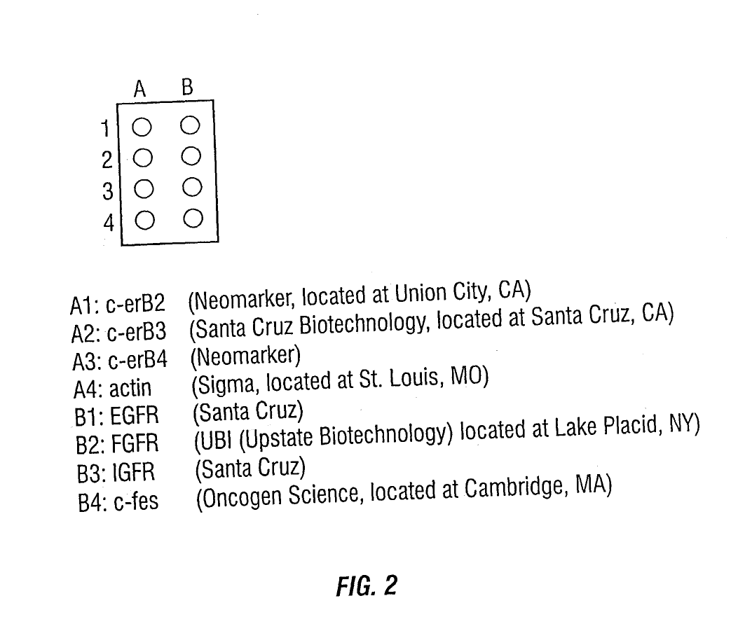

A nitrocellulose membrane (2×4 CM Protran BA85, S&S) in soaked in phosphate buffered saline (PBS). Antibodies specific for the target proteins of interest (listed below) are diluted 1:100 in PBS. The diluted antibodies are spotted in 25 ul aliquots onto a nitrocellulose membrane through a well created by a dot blot apparatus according to the position indicated below. The membrane is washed in PBS, and kept in PBS containing 0. 1% thimerosal. The immobilized proteins are listed as follows (see FIG. 2):

| | |
|---|---|
| A1: c-erB2 | (Neomarker, located at Union City, CA) |
| A2: c-erB3 | (Santa Cruz Biotechnology, located at Santa Cruz, CA) |
| A3: c-erB4 | (Neomarker) |
| A4: actin | (Sigma, located at St. Louis, MO) |
| B1: EGFR | (Santa Cruz) |
| B2: FGFR | (UBI (Upstate Biotechnology) located at Lake Placid, NY) |
| B3: IGFR | (Santa Cruz) |
| B4: c-fes | (Oncogen Science, located at Cambridge, MA) |

2. Sample Preparation

Cell lines NIH3T3, NIH3T3/EGFR and NIH3T3/c-erB2 were cultured in DMEM containing 5% FBS to 70% confluence. Cells were harvested and sonicated in lysis buffer (50 mM Tris.Cl, pH 7.4, 150 mM NaCl, 1 mM EGTA, 1 mM Na3VO4, 100 nM okadaic acid, 1 mM PMSF, 1 ug/ml aprotinin, leupeptin and pepstatin, and 1% NP40). Cell lysate was centrifuged at 14,000 rpm for 5 min, and supernatant was diluted to protein concentration of 1 mg/ml.

3. Hybridization with Protein Tyrosine Kinase (PTK) Membrane Protein Array

PTK protein array membranes were incubated with DetectorBlock solution (KPL, Gaitheburgh, Md.) for 1 hr. Rinse membrane with TBST (50 mM Tris.Cl, pH 7.0, 150 mM NaCl, 0.1% Tween 20). 100 ul cell lysate was dropped on plastic wrap, then the membrane PTK was placed down facing the cell lysate. The membrane was covered with plastic wrap to prevent evaporation, and kept at room temperature for 1 hr. The membrane was washed with TBST for 10 min 3 times. The membrane was incubated with 1:1000 diluted anti-phosphotyrosine-peroxidase (anti-P-Try-POD) (Boehringer Mannheim) in TBST for 45 min. The membrane was washed with TBST for 10 min 3 times. The membrane was incubated with ECL reagent (Amersham) for 1 min, then wrapped with plastic wrap. The membrane was exposed to X-ray film. The results indicated a positive at position A1 on the membrane that was hybridized with cell lysate prepared from c-erb2 transfected NIH3T3 cells, indicating that a tyrosine phosphorylated c-erb2 target protein was in that cell lysate.

(1) membrane 1 was hybridized with cell lysate prepared from NIH3T3 cells;

(2) membrane was hybridized with cell lysate prepared from EGFRtransfected NIH3T3 cells;

(3) membrane was hybridized with cell lysate prepared from c-erb2 transfected NIH3T3 cells. Membrane (3) indicated a signal at a location on the upper left-hand corner of the membrane at position A1.

4. Testing ErbB Family Receptor Protein Arrays

To determine the optimal conditions for a protein array for the ErbB family of receptors (also called the EGF family of receptors), each ErbB receptor can be tested to establish the conditions for the entire array in order to optimize the specificity for the ErbB protein array. A solid support is spotted with capture molecules (antibodies) specific for the members of the ErbB receptor family (e.g. EFGR, ErbB-2, ErbB-3, ErbB-4). IMDA-468 cell line (Kassis J, et al., Clin Cancer Res 1999 Aug;5(8):2251-60; Reddy KB, et al., Int J Oncol 1999 Aug;15(2):301-6) which expresses both EGFR and ErbB-3 receptor is treated with EGF to stimulate and derive tyrosine-phosphorylated EGFR while ErbB-3 remains unphosphorylated. To evaluate the specificity the protein array assay for EGFR, the cell lysate from MDA-468 cells is incubated with the ErbB protein array. The specific detection of EGFR using the protein array assay should ensure no signals obtained from the ErbB-2 receptor, ErbB-3 and ErbB-4 spots and a strong signal from EGFR spot.

A 32D cell line (Ruggiero, M et al., 1991, FEBS, 291:203) transfected with ErbB-2 is stimulated by heregulin to derive tyrosine-phosphorylated ErbB-2. Specific detection of ErbB-2 using the protein array assay ensures no signals obtained from the EGFR receptor, ErbB-3 and ErbB-4 spots and a strong signal from ErbB-2 spot.

A 32D cell line transfected with ErbB-3 is stimulated by heregulin to derive tyrosine-phosphorylated ErbB-3. Specific detection for ErbB-3 using the protein array assay ensures no signals obtained from the EGFR receptor, ErbB-2 and ErbB-4 spots and a strong signal from ErbB-3 spot.

A 32D cell line transfected with ErbB-4 is stimulated by heregulin to derive tyrosine-phosphorylated ErbB-4. Specific detection for ErbB-4 using the protein array assay ensures no signals obtained from the EGFR receptor, ErbB-2 and ErbB-3 spots and a strong signal from ErbB-4 spot.

The dosage response of tyrosine phosphorylation of ErbB family receptors can be detected by the protein array for that family and compared with dosage responses detected by Westernblot analysis. MDA-468 cells are stimulated with EGF at 3000, 1000, 333, 111, 37, 12, 4 or 0 pM separately. An aliquot of the lysate from each sample is prepared and incubated with the ErbB protein array to determine the level of tyrosine phosphorylation. An equal aliquot of the lysate is immunoprecipitated with EGRF antibody followed by Westernblot to determine for the level of tyrosine phosphorylation on EGFR. This process is repeated for each other target protein in the array (ErbB-2, 3, & 4) using the 32D cell line, stimulation with heregulin at 3000, 1000, 333, 111, 37, 12, 4 or 0 pM, and in each case an aliquot of the lysate from each sample is prepared and incubated with the ErbB protein array to determine a level of tyrosine phosphorylation, and these results are compared and correlated with results from both Westerblot analysis and the proposed protein array analysis.

A screen of 12 tumor cell lines is established for ErbB receptor family activation using an ErbB protein array to compare the cell lines for the presence of the tyrosine phosphorylated receptors. Twelve well-characterized tumor cell lines are used (MDA-453, BT-474, MDA-361, N87, MCF-7, SKBr3, MDA-468, A431, MDA-231, LCC6, SKOv3 and MCF10A (available at the ATCC; Yang D et al., 1998, Clinical Cancer Research 4:993-1004). These cells are stimulated with EGF and heregulin. The lysate from each of these cell lines is used to incubate with the ErbB protein array to determine the individual level of tyrosine phosphorylation of four ErbB receptors. The level of tyrosine phosphorylation of these ErbB receptors is also determined by Westernblot analysis. These comparative data are then correlated.

A cell line from group of 12 cancer cell lines tested above is selected that has the highest amount of activation of the ErbB family receptors in the ErbB protein array assay. This cell line is selected for screening for ErbB receptor activation inhibitors. Multiple different potential inhibitors of ErbB receptor activation are selected for screening, and the cell line is administered these test drugs. The cell lysate contacts an ErbB receptor array and a tyrosine phosphorylation antibody detects the identity and levels of phosphorylated target proteins that are captured by the capture molecules on the solid support. Candidate molecules for inhibiting ErbB receptor activation are then selected. Some candidates may appear to work most effectively on some but not all ErbB receptors, and thus cocktails of the test drugs may also be tested.

Since modifications will be apparent to those of skill in this art, it is intended that this invention be limited only by the scope of the appended claims.

1. A method for detecting protein modification of a target protein in a sample, which method comprises:
a) contacting a sample containing or suspected of containing a target protein with a capture molecule immobilized on a solid support, said capture molecule is capable of specifically binding to said target protein, whereby said target protein is immobilized on said solid support; and
b) assessing modification status and/or identity of said immobilized target protein.

2. The method of claim 1, wherein a plurality of target proteins is contacted with the immobilized capture molecule simultaneously.

3. The method of claim 2, wherein the plurality of target proteins comprises a group of structurally and/or functionally related proteins.

4. The method of claim 1, wherein a target protein is contacted with a plurality of immobilized capture molecules simultaneously.

5. The method of claim 1, wherein a plurality of target proteins is contacted with a plurality of immobilized capture molecules simultaneously.

6. The method of claim 1, wherein the capture molecule is capable of specifically binding to both a modified and an unmodified forms of a target protein.

7. The method of claim 1, wherein the capture molecule is capable of specifically binding to a modified form of the target protein but is not capable of specifically binding to an unmodified form of the target protein.

8. The method of claim 1, wherein the capture molecule is an antibody.

9. The method of claim 1, wherein the solid support is selected from the group consisting of silicon, plastic, nylon, glass, ceramic, photoresist, rubber or polymer support.

10. The method of claim 1, wherein the solid support comprises a flat support, a set of sticks, or a set of beads.

11. The method of claim 10, wherein the flat support comprises a slide, a chip, a filter, or a membrane.

12. The method of claim 1, wherein the protein modification is selected from the group consisting of phosphorylation, acetylation, methylation, ADP-ribosylation, addition of a polypeptide side chain, addition of a hydrophobic group, and addition of a carbohydrate.

13. The method of claim 12, wherein the phosphorylation is on an amino acid residue selected from the group consisting of tyrosine, serine, threonine and histidine.

14. The method of claim 12, wherein the addition of a polypeptide side chain is the addition of ubiquitin.

15. The method of claim 12, wherein the addition of a hydrophobic group is the addition of a fatty acid, addition of an isoprenoid, or addition of a glycosyl-phosphatidyl inositol anchor.

16. The method of claim 15, wherein the fatty acid is myristate or palmitate.

17. The method of claim 15, wherein the isoprenoid is famesyl or genranylgenranyl.

18. The method of claim 12, wherein the carbohydrate group comprises glycosyl.

19. The method of claim 6, wherein the modification status of the immobilized target protein is assessed by contacting the immobilized target protein with a detection molecule that is capable of specifically binding to the modified target protein but is not capable of specifically binding to the unmodified target protein itself.

20. The method of claim 6, wherein the modification status of the immobilized target protein is determined by a physical or chemical means.

21. The method of claim 20, wherein the physical or chemical means comprises chemical or radioisotopic label of the protein modification moiety.

22. The method of claim 20, wherein the physical or chemical means is selected from the group consisting of chromatographic, electrophoretic, protein sequencing, mass spectrometry and NMR means for detecting the protein modification moiety.

23. The method of claim 7, wherein the identity of the immobilized target protein is assessed by contacting the immobilized target protein with a detection molecule that is capable of specifically binding to the unmodified target protein itself but is not capable of specifically binding to the modified target protein.

24. The method of claim 1, wherein the sample is a biological sample.

25. The method of claim 1, wherein the target protein is involved in a biological pathway, belongs to a group of proteins with identical or similar biological function, expressed in a stage of cell cycle, expressed in a cell type, expressed in a tissue type, expressed in an organ type, expressed in a developmental stage, a protein whose expression and/or activity is altered in a disease or disorder type or stage, or a protein whose expression and/or activity is altered by drug or other treatments.

26. A method for identifying biologically distinguishable marker(s) associated with a biosample, which comprises:
1) assessing protein modification profile of a biosample through the method of claim 1;
2) assessing protein modification profile of a comparable control biosample through the method of claim 1; and
3) comparing the protein modification profile obtained in step 1) with the protein modification profile obtained in step 2) to identify biologically distinguishable protein modification profile marker(s) associated with said biosample.

27. A kit for detecting protein modification, which kit comprises:
a) a capture molecule immobilized on a solid support, said capture molecule is capable of specifically binding to a target protein; and
b) means for assessing modification status and/or identity of said target protein.

28. The kit of claim 27, wherein a plurality of capture molecules is immobilized on the solid support, each of said capture molecules is capable of specifically binding to a member protein of a group of structurally and/or functionally related target proteins.

29. The kit of claim 27, wherein the capture molecule is capable of specifically binding to both a modified and an unmodified forms of a target protein.

30. The kit of claim 27, wherein the capture molecule is capable of specifically binding to a modified form of the target protein but is not capable of specifically binding to an unmodified form of the target protein.

31. The kit of claim 27, wherein the capture molecule is an antibody.

32. The kit of claim 27, wherein the protein modification is selected from the group consisting of phosphorylation, acetylation, methylation, ADP-ribosylation, addition of a polypeptide side chain, addition of a hydrophobic group, and addition of a carbohydrate.

33. The kit of claim 29, wherein the modification status of the immobilized target protein is assessed by contacting the immobilized target protein with a detection molecule that is capable of specifically binding to the modified target protein but is not capable of specifically binding to the unmodified target protein itself.

34. The kit of claim 29, wherein the modification status of the immobilized target protein is determined by a physical or chemical means.

35. The kit of claim 30, wherein the identity of the immobilized target protein is assessed by contacting the immobilized target protein with a detection molecule that is capable of specifically binding to the unmodified target protein itself but is not capable of specifically binding to the modified target protein.

36. The kit of claim 27, further comprising:
a) instructions for using the kit;
b) reagents and buffers; and/or
c) a container(s) for the kit contents.

37. An array of protein capture molecules, which array comprises:
a) a solid support; and
b) a plurality of capture molecules immobilized on said solid support, wherein each of said molecules is capable of specifically binding to both a modified and an unmodified form of a member protein of a plurality of target proteins.

38. The array of claim 37, wherein the plurality of target proteins comprises a group of structurally and/or functionally related proteins.

39. The array of claim 38, wherein the modified and unmodified forms of the same target protein have different biological activities.

40. The array of claim 38, wherein the modified and unmodified forms of the same target protein represent different physiological conditions or biological statuses.

41. The array of claim 40, wherein the array is used to identify pathway activation.

42. The array of claim 40, wherein the array is used to identify activation of a group of structurally and/or functionally related protein.

43. The array of claim 40, wherein the array is used to generate a modification profile correlated to a physiological condition, drug treatment and disease.

44. The array of claim 40, wherein the array is used to identify a physiological or pathological status.

45. The array of claim 40, wherein the array is used to record biological perturbation caused by drug and other treatment.

46. An array of protein capture molecules, which array comprises:
a) a solid support; and
b) a plurality of capture molecules immobilized on said solid support, wherein each of said molecules is capable of specifically binding to an epitope generated by a specific modification moiety of a modified protein.

47. The array of claim 46, wherein the modified protein is Rb.

48. An array of enzyme substrates, which array comprises:
a) a solid support; and
b) a plurality of substrates immobilized on said solid support, wherein each of said substrates is a substrate of a member enzyme of a group of structurally and/or functionally related enzymes.

49. The array of claim 48, wherein at least one of the member enzymes catalyzes a protein modification reaction.

50. A kit for detecting enzymatic activity, which kit comprises:
a) the array of claim 48; and
b) means for assessing activity of each of the member enzymes.

51. A method for detecting enzymatic activity in a sample, which method comprises:
a) contacting a sample containing or suspected of containing a group of structurally and/or functionally related target enzymes with a plurality of substrates immobilized on a solid support, wherein each of said substrates is a substrate of a member enzyme of said group of target enzymes under conditions suitable for said target enzymes to catalyze enzymatic reactions involving said immobilized substrates; and
b) assessing enzymatic activities of said target enzymes.

52. The method of claim 51, wherein at least one of the target enzymes catalyzes a protein modification reaction.
